(19) United States
(12) Patent Application Publication
(10) Pub. No.: US 2025/0242130 A1
(43) Pub. Date: Jul. 31, 2025

(54) GRIPPING DEVICES FOR CATHETER ASSEMBLIES

(71) Applicant: ABIOMED, Inc., Danvers, MA (US)

(72) Inventors: Christopher W. Sheils, Danvers, MA (US); Christopher Theodore Bazdanes, Danvers, MA (US); Jeremy Yigal Nezaria, Danvers, MA (US); Iain Zwiebel, Danvers, MA (US); Jennifer Selingo, Danvers, MA (US); Lindsay Baxter, Danvers, MA (US)

(73) Assignee: ABIOMED, Inc., Danvers, MA (US)

(21) Appl. No.: 19/037,103

(22) Filed: Jan. 25, 2025

Related U.S. Application Data
(60) Provisional application No. 63/742,671, filed on Jan. 7, 2025.
(60) Provisional application No. 63/726,997, filed on Dec. 2, 2024.
(60) Provisional application No. 63/625,779, filed on Jan. 26, 2024.

(51) Int. Cl. A61M 25/01; A61M 25/02; A61M 25/06; A61M 39/06; A61M 60/13; A61M 60/17; A61M 60/216; A61M 60/865

(52) U.S. Cl. CPC A61M 25/0136; A61M 25/02; A61M 39/06; A61M 60/13; A61M 60/17; A61M 60/216; A61M 60/865; A61M 2025/0681; A61M 2205/586

(57) ABSTRACT

Gripping devices for catheter assemblies, such as, intracardiac blood pump assemblies are provided.

### CROSS REFERENCE TO RELATED APPLICATIONS

This application claims the priority of and benefit from U.S. Provisional Application No. 63/625,779, filed Jan. 26, 2024, U.S. Provisional Application No. 63/726,997, filed Dec. 2, 2024, and U.S. Provisional Application No. 63/742,671, filed Jan. 7, 2025, each of which is incorporated by reference herein.

### TECHNICAL FIELD

The present technology relates to gripping devices for catheter assemblies, such as, intracardiac blood pump assemblies.

### BACKGROUND

Catheter assemblies, such as, intracardiac blood pump assemblies can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

Catheter assemblies, such as, intracardiac blood pump assemblies can be introduced into the heart by a catheterization procedure. For example, with respect to an intracardiac blood pump assembly inserted into the left heart, an introducer sheath assembly may be inserted into the femoral artery through an arteriotomy to gain access to the artery and create an insertion path. A placement guidewire can be advanced into the artery along the insertion path. After the guidewire has been inserted into the artery, the pump assembly can be advanced over the guidewire and into the patient. Alternatively, the pump assembly can be inserted directly into the artery without a guidewire. The blood pump of the assembly can be inserted via a catheterization procedure through the femoral artery, into the ascending aorta, across the aortic valve and into the left ventricle. When deployed in the left heart, the pump assembly pulls blood from the left ventricle and expels blood into the ascending aorta.

### BRIEF SUMMARY

The present technology relates to gripping devices for catheter assemblies, such as, intracardiac blood pump assemblies.

In one aspect of the present technology, a device for gripping a portion of a catheter of a catheter assembly is provided. The device comprises a longitudinal axis, a housing, a securing mechanism, and a grippable handle. The housing comprises a proximal end and a distal end and extends along the longitudinal axis from the proximal end to the distal end of the housing. The housing is configured to receive the catheter through an interior of the housing. The securing mechanism is configured to be selectively engaged to restrict or permit movement of the catheter through the housing. The grippable handle comprises a proximal end, a distal end, an exterior surface, and an interior surface defining an interior lumen. The grippable handle extends along the longitudinal axis from the proximal end to the distal end of the grippable handle. The interior lumen extends along the longitudinal axis and is configured to receive the portion of the catheter. The distal end of the grippable handle is rotatably coupled to the proximal end of the housing such that the grippable handle is rotatable relative to the housing about the longitudinal axis. The grippable handle is configured to deform in response to a compression force applied to the grippable handle such that the interior surface grips the portion of the catheter to permit the grippable handle to transfer a torque force to the portion of the catheter.

In some aspects of the device, the grippable handle is configured to deform in response to a compression force applied to the exterior surface of the grippable handle.

In some aspects of the device, the device further comprises an actuator configured to apply the compression force to the grippable handle.

In some aspects of the device, the distal end of the housing is configured to couple with a fixation device that is attachable to a patient.

In some aspects of the device, the distal end of the housing is configured to couple with the fixation device via a hemostasis valve disposed between the housing and the fixation device.

In some aspects of the device, the distal end of the housing includes a cavity configured to receive a proximal portion of the hemostasis valve.

In some aspects of the device, the distal end of the grippable handle is coupled to the proximal end of the housing such that the grippable handle is translatable relative to the housing along the longitudinal axis.

In some aspects of the device, the grippable handle is translated along the longitudinal axis and the interior surface grips the portion of the catheter, the grippable handle is configured to transfer a push or a pull force to the catheter.

In some aspects of the device, the device further comprises a first coupling mechanism configured to rotatably couple the distal end of grippable handle to the proximal end of the housing.

In some aspects of the device, the first coupling mechanism is configured to couple the distal end of the grippable handle to the proximal end of the housing such that the grippable handle is translatable relative to the housing along the longitudinal axis.

In some aspects of the device, the device further comprises a second coupling mechanism configured to rotatably couple the proximal end of the grippable handle to a protective sleeve of the catheter assembly.

In some aspects of the device, the grippable handle is made of a rubber or silicone material.

In some aspects of the device, a diameter of the interior lumen is larger than an outer diameter of the catheter when the grippable handle is not compressed.

In some aspects of the device, the device is slidable over the exterior of the catheter when the grippable handle is not compressed and the securing mechanism is engaged to permit movement of the catheter through the housing.

In one aspect of the present technology, a catheter assembly is provided. The catheter assembly, comprises a catheter and a gripping device. The gripping device comprises a proximal end, a distal end, an exterior surface, and an interior surface defining an interior lumen. The interior lumen extends from the proximal end to the distal end and is configured to receive a portion of the catheter. The gripping device is configured to deform in response to a compression force applied to the exterior surface of the gripping device such that the interior surface grips the portion of the catheter to permit the gripping device to transfer a torque, push, and/or pull force to the portion of the catheter.

In some aspects of the catheter assembly, the gripping device is configured to deform in response to a compression force applied to the exterior surface of the gripping device.

In some aspects of the catheter assembly, the catheter assembly further comprises a sleeve disposed over the catheter.

In some aspects of the catheter assembly, the sleeve and the portion of the catheter extend through the interior lumen such that the gripping device is disposed over the sleeve.

In some aspects of the catheter assembly, the gripping device is coupled to the sleeve. In some aspects of the catheter assembly, the gripping device is joined to the sleeve.

In some aspects of the catheter assembly, gripping device is coupled to one of a proximal end or a distal end of the sleeve.

In some aspects of the catheter assembly, the gripping device is coupled to a portion of the sleeve between a proximal end and a distal end of the sleeve.

In some aspects of the catheter assembly, the gripping device is disposed interior to the sleeve and exterior to the catheter.

In some aspects of the catheter assembly, the interior lumen is dimensioned such that that gripping device is slidable over the exterior of the catheter.

In some aspects of the catheter assembly, the interior lumen is dimensioned such that the gripping device is slidable over the exterior of the catheter when the gripping device is not compressed.

In some aspects of the catheter assembly, the exterior surface of the gripping device has an ergonomic shape.

In some aspects of the catheter assembly, the gripping device is made of a rubber or silicone material.

In some aspects of the catheter assembly, the catheter assembly is an intracardiac blood pump assembly.

In some aspects of the catheter assembly, the catheter assembly further comprises a blood pump comprising a proximal end and a distal end, wherein a distal end of the catheter is coupled to a proximal end of the blood pump.

### BRIEF DESCRIPTION OF THE DRAWINGS

FIG. 1A illustrates an exemplary blood pump portion of an intracardiac blood pump assembly for left heart support in accordance with aspects of the present technology.

FIGS. 1B and 1C illustrate additional components of the exemplary intracardiac blood pump assembly of FIG. 1A in accordance with aspects of the present technology.

FIG. 2 illustrates a portion of the exemplary intracardiac blood pump assembly of FIG. 1A including a securement device in accordance with aspects of the present technology.

FIG. 3 shows an enlarged view of a blood pump and repositioning sheath assembly using an exemplary button-actuated securement device, in accordance with aspects of the present technology.

FIG. 4 shows an isometric view of a three-dimensional model of the exemplary button-actuated securing device of FIG. 3.

FIG. 5 shows a line-drawing of the exemplary button-actuated securing device of FIG. 3.

FIG. 6 shows an exploded view of the exemplary button-actuated securing device of FIG. 3.

FIG. 7 shows a cross-sectional profile view of the exemplary button-actuated securing device of FIG. 3.

FIG. 8 shows an enlarged view of a blood pump and repositioning sheath assembly using an exemplary button-actuated securement device, in accordance with aspects of the present technology.

FIG. 9 shows an enlarged view of a blood pump and repositioning sheath assembly using an exemplary button-actuated securement device, in accordance with aspects of the present technology.

FIGS. 10A-10D are exploded views of features of the apparatus of FIG. 6, but with a different retention pin design and a different design for the second bore of the button.

FIG. 11 illustrates a securement device configured to grip a portion of a catheter of a blood pump assembly in accordance with aspects of the present technology.

FIG. 12 illustrates a securement device configured to grip a portion of a catheter of a blood pump assembly in accordance with aspects of the present technology.

FIG. 13 illustrates a securement device with a grippable handle configured to grip a portion of a catheter of a blood pump assembly in accordance with aspects of the present technology.

FIG. 14 illustrates a securement device with a grippable handle configured to grip a portion of a catheter of a blood pump assembly in accordance with aspects of the present technology.

FIGS. 15A-15C illustrate a securement device with a grippable handle configured to grip a portion of a catheter of a blood pump assembly in accordance with aspects of the present technology.

FIGS. 16A-16B illustrate the securement device of FIGS. 15A-15C further including a coupling component coupled to the proximal end of the grippable handle of the securement device of FIGS. 15A-15C in accordance with aspects of the present technology.

FIG. 17 illustrates a gripping device for gripping a portion of a catheter of a blood pump assembly in accordance with aspects of the present technology.

FIG. 18 illustrates a gripping device for gripping a portion of a catheter of a blood pump assembly in accordance with aspects of the present technology.

FIG. 19 illustrates a gripping device for gripping a portion of a catheter of a blood pump assembly in accordance with aspects of the present technology.

### DETAILED DESCRIPTION

Aspects of the present technology are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

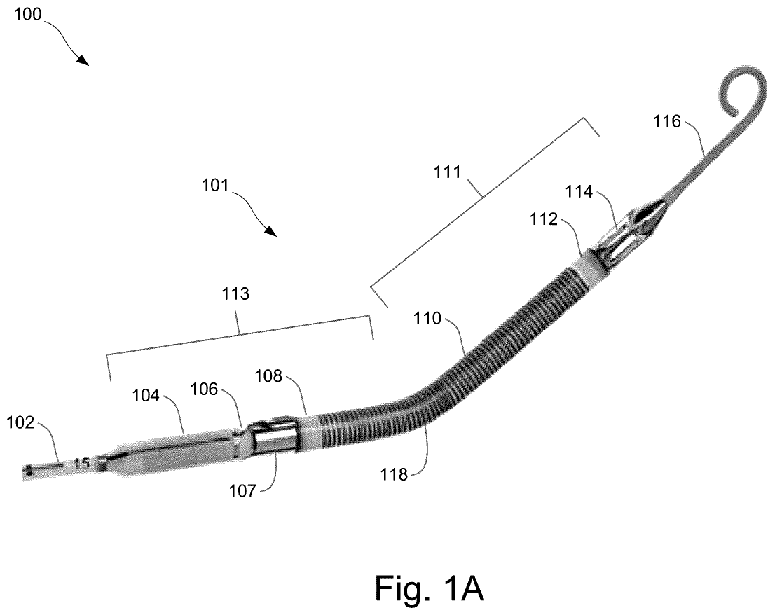

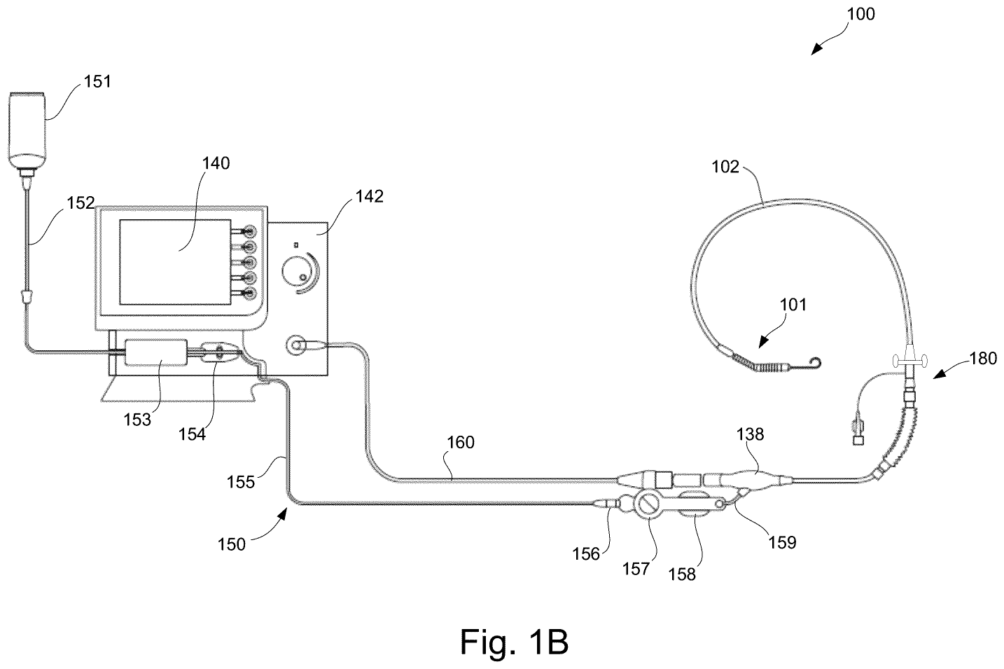

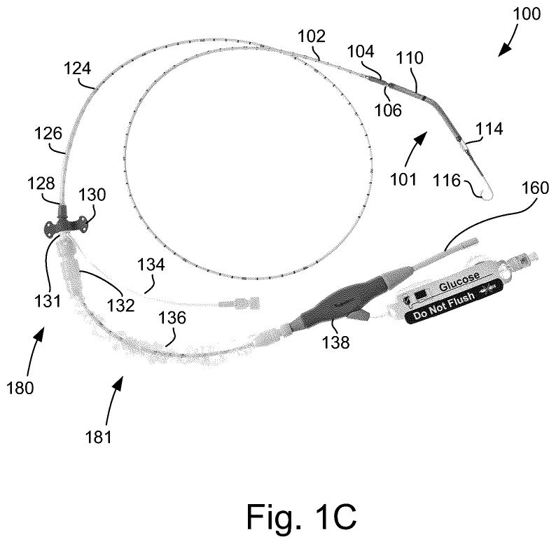

FIG. 1A depicts a blood pump portion 101 of an exemplary intracardiac blood pump assembly 100 adapted for left heart support, in accordance with aspects of the present technology. As shown in FIG. 1A, an intracardiac blood pump assembly 100 adapted for left heart support may include an elongate catheter 102, a motor housing 104, a cannula 110, a blood flow inlet 114 arranged at or near the distal end 112 of the cannula 110, a blood flow outlet 106 arranged at or near the proximal end 108 of the cannula 110, and an optional atraumatic extension 116 arranged at the distal end of the blood inflow cage 114. In one aspect, the inlet 114 is configured as a blood inflow cage and the outlet 106 is configured as a blood outflow cage. The motor 104, cannula 110, blood inflow cage 114, blood outflow cage 106 form a blood pump portion 101 of assembly 100. The blood pump portion 101 has a first portion 111 and a second portion 113. Portion 113 is proximal to portion 111. In one aspect, pre-formed bend 118 is disposed between the first portion 111 and the second portion 113.

In some aspects of the present technology, motor housing 104 houses a motor (not shown) that is configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an onboard motor in motor housing 104, the impeller may instead be coupled to an elongate drive shaft (or drive cable) which is driven by a motor located external to the patient.

Catheter 102 may house electrical lines coupling the motor in motor housing 104 to one or more electrical controllers and/or sensors. Alternatively, where the impeller is driven by an external motor, an elongate drive shaft may pass through catheter 102. Catheter 102 may also include a purge fluid conduit, a lumen configured to receive a guidewire, one or more optical fibers (e.g., for sensing pressure), etc.

The blood inflow cage 114 may include one or more apertures or openings configured to allow blood to be drawn into cannula 110 when the motor in motor housing 104 is operating. Likewise, blood outflow cage 106 may include one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, as shown in the exemplary arrangement of FIG. 1A, the cannula 110 may have a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 may assist with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials, or may be composed of the same material with the proximal and distal sections being treated to provide different stiffnesses.

Notwithstanding the foregoing, as mentioned above, atraumatic extension 116 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As shown in FIG. 1A, the distal end of catheter 102 is coupled to the proximal end of proximal portion 113 (e.g., to motor housing 104). The proximal portion of catheter 102 is further coupled to additional components of blood pump assembly 100, as shown in FIG. 1B.

In this regard, as shown in FIG. 1B, in addition to blood pump 101 and catheter 102, the blood pump assembly 100 may further include a purging device or assembly 150, a controller 142 (e.g., an Automated Impella Controller® from Abiomed, Inc., Danvers, MA), a display 140, a connector cable 160, a plug 138, and a repositioning unit 180. In some aspects, controller 142 includes display 140. Controller 142 comprises one or more processors. Controller 142 monitors and controls blood pump 101. During operation, purging device 150 delivers a purge fluid to blood pump 101 through catheter tube 102 to prevent blood from entering the motor (not shown) within motor housing 104. In some implementations, the purge fluid comprises a dextrose solution (e.g., 5% dextrose in water with 25 or 50 IU/mL of heparin). Connector cable 160 may provide electrical and/or optical connection(s) between blood pump 101 and controller 142. Plug 138 connects catheter tube 102, purging device 150, and connector cable 160. In some embodiments, plug 138 includes a memory for storing operating parameters in case the patient needs to be transferred to another controller 142. As will be described in greater detail below, repositioning unit 180 may be used to (position and) reposition blood pump 101 within a patient.

As shown, purging device 150 comprises a reservoir 151, purge fluid supply line 152, a purge cassette 153, a purge disc 154, purge tubing 155, a check valve, a pressure reservoir 157, an infusion filter 158, and a sidearm 159. Reservoir 151 may, for example, be a bag or a bottle. A purge fluid is stored in reservoir 151. A purge fluid spike at the end of purge fluid supply line 152 may be used to puncture reservoir 151 and connect the purge fluid in reservoir 151 to purge fluid supply line 152. Purge fluid supply line 152 carries the purge fluid from reservoir 151 to purge cassette 153. Purge tubing 155 carries the purge fluid from purge cassette 153 to blood pump 101.

Purge cassette 153 controls how the purge fluid in reservoir 151 is delivered to blood pump 101 and the flow path of the purge fluid from reservoir 151 to blood pump 101. For example, purge cassette 153 may include one or more valves (e.g., purge path diverters) for controlling a pressure and/or flow rate of the purge fluid. In addition to containing the components for delivering the purge fluid, purge cassette 153 also maintains the pressure barrier between the blood and the motor of blood pump 101 to prevent blood from entering the motor. Purge cassette 153 may contain a rack and pinion which is attached to a piston. Purge disc 154 includes one or more measuring devices, such as pressure sensors (e.g., a pressure-sensing diaphragm) for measuring purge pressure of the purge fluid at blood pump 101. Controller 142 is connected to purge cassette 153 and purge disc 154. Purge disc 154 transmits pressure to controller 142 based on the purge pressure in purge tubing 155. A sensor in controller 142 measures the pressure so that it can be displayed on screen 140. Controller 142 may include a stepper motor. A tic (or step) represents stepper motor positions in units of microsteps, which are also called pulses. In some embodiments, a purge pressure/purge flow curve algorithm is deployed by the controller 142 along with the number of steps/minute of the stepper motor, and pressure measurement by purge disc 154 to calculate the corresponding purge flow rate.

As described above, purge tubing 155 provides a fluidic connection passing through purge cassette 153 to blood pump 101. In some embodiments, a Y-connector and/or a yellow Luer connector are also provided, which facilitate a continuous fluid path or purge fluid path. Y-connector is an adapter that connects purge tubing 155 to blood pump 101. Yellow Luer connector connects purge tubing 155 to a check valve (yellow luer lock) on blood pump 101. Pressure reservoir 157 provides additional filling volume during a purge fluid change. In some embodiments, pressure reservoir 157 includes a flexible rubber diaphragm that provides the additional filling volume by means of an expansion chamber. Infusion filter 158 helps prevent bacterial contamination and air from entering catheter tube 102. Sidearm 159 provides a fluidic connection between infusion filter 158 and plug 138.

During operation, controller 142 receives measurements from purge disc 154 and controls the stepper motor's number of ticks to control the purge pressure. In some embodiments, during operation, purge cassette 153 is placed in controller 142 and connected with blood pump 101. As noted above, controller 142 controls and measures purge pressure and calculates purge flow rate via purge cassette 153 and/or purge disc 154. Controller 142 may also control the purge fluid supply. During operation, after exiting purging device 150 through sidearm 159, the purge fluid is channeled through purge lumens (not shown) within catheter tube 102 and plug 138. Sensor cables (not shown) within catheter tube 102, connector cable 160, and plug 138 provide an electrical connection between purge disc 154 and controller 142. Motor cables (not shown) within catheter tube 102, connector cable 160, and plug 138 provide an electrical connection between the motor within motor housing 104 and controller 142. During operation, controller 142 receives measurements from purge disc 154 through the sensor cables and controls the electrical power delivered to the motor within motor housing 104 through the motor cables. By controlling the power delivered to the motor within motor housing 104, controller 142 can control the speed of the motor within motor housing 104. In some embodiments, controller 142 includes safety features to prevent air from entering purge tubing 155. Controller 142 may include (or be in communication with) circuitry for monitoring the motor current for drops in current indicating air in the line. Controller 142 may include or be configured to generate warning sounds, lights or indicators to alert an operator of certain detected conditions, such as, but no limited to, pump position, suction events at the inlet, and disconnects or breaks in purge tubing 155 which may result in the introduction of air to the line.

In some aspects, assembly 100 may include one or more sensors or measurements devices configured to communicate with controller 142 to provide information associated with the operation of assembly 100 or associated with a patient. In one aspect, assembly 100 may include an optical fiber (disposed through one or more of cable 160, plug 138, and catheter 102) that forms a pressure sensitive surface at its distal end. The pressure sensitive surface forms a pressure sensor which may be added to blood pump 101 near inlet area 114. The pressure sensor is configured to measure a left ventricular blood pressure. Assembly 100 may implement multiple such pressure sensors (or other sensor types) at different locations (e.g., on or in pump 101, catheter 102, plug 138, etc.) throughout assembly 100 and controller 142 may be configured to perform different steps based on the information received for the sensors. In some aspects, additional sensor cables may be disposed within catheter tube 102, connector cable 160, and plug 138 to provide an electrical and/or optical connection between the one or more additional measuring devices and controller 142. As yet another example, one or more components of blood pump assembly 100 may be separated. For example, display 140 may be incorporated into another device in communication with controller 142 (e.g., wirelessly or through one or more electrical cables).

Display 140 is controllable by controller 142 to display useful information to the user of the blood pump assembly 100. For example, display 140 may be controlled to display many different types of information such as the characteristics of the blood pump assembly (e.g., blood pump type, serial number, software version, etc.), operation of the blood pump assembly (e.g., present blood pump speed (performance) setting, blood pump flow measurements, purging device measurements, a status indicator, sensor measurements, blood pump position detections and indications, etc.). Some of this information may be obtained from purge disc 154 or any of the other sensors described above or that may be used with assembly 100. Display 140 can also provide notifications to the user. For example, a notification may serve as an alert and include a statement describing the cause of the alert. In some embodiments, display 140 may be a touchscreen and a user may switch between screens by tapping button labels on display 140. In some embodiments, a user may use a separate input device, such as a mouse or a keyboard, to switch between screens.

As described above, assembly 100 may include a repositioning unit 180 for repositioning the blood pump 101 within the patient. For example, FIG. 1C shows an exemplary implementation of blood pump assembly 100 with a repositioning unit or assembly 180, in accordance with aspects of the present technology.

In one aspect, the repositioning unit 180 may include repositioning sheath 126, fixation device or butterfly 130, hemostasis valve 131, securement device 132, and protective sleeve 136. In this aspect, between handle 138 and securement device 132, the catheter 102 is enclosed within a protective sleeve 136. Protective sleeve 136 may be configured to prevent contamination of catheter 102 as it is advanced in the distal direction for insertion into the patient's vasculature. Protective sleeve 136 may be comprised of any suitable material, and may be secured at its proximal and distal ends in any suitable manner. The distal end of protective sleeve 136 may be coupled to securement device 132. In the example of FIG. 1C, securement device 132 is coupled at its distal end to a hemostasis valve 131, which in turn is attached to butterfly 130. Hemostasis valve 131 may be integrated with butterfly 130, or may be removably coupled thereto. In addition, hemostasis valve 131 and securement device 132 may be incorporated into a single unit. Butterfly 130 is coupled at its distal end to the proximal end 128 of repositioning sheath 126. The distal end of repositioning sheath 126 is indicated with reference numeral 124. Repositioning sheath 126 is configured with a lumen sized to allow passage of at least catheter 102, but otherwise may have any suitable length and construction.

Generally, a blood pump, such as blood pump 101, may initially be inserted into the patient's vasculature via an introducer sheath assembly. Once the blood pump 101 has been inserted into the patient's vasculature, the operator may advance the blood pump 101 to its desired location in the body (e.g., left heart or right heart). As the catheter 102 advances further into the patient's vasculature, the distal end 124 of repositioning sheath 126 may be inserted into the introducer sheath assembly which acts as a conduit for repositioning sheath 126 to enter the patient's vasculature. In some aspects, the introducer sheath may be removed thereafter, for example, in the case of a tear away design, by tearing the introducer along its length. However, in other aspects, where introducer sheath assembly is an expandable design, it may remain in the body, either surrounding some or all of the repositioning sheath 126, or, in cases where the repositioning sheath 126 remains outside of the body, surrounding catheter 102.

Once repositioning sheath 126 has been fully inserted, the operator may secure it to the patient at or near the insertion site using butterfly 130. In that regard, butterfly 130 may be affixed to the patient (e.g., using adhesives or sutures) in order to secure the repositioning sheath 126 and securement device 132 relative to the patient. Thereafter, once the blood pump has been advanced to the desired location within the patient's body, the operator may use securement device 132 to restrict further movement of the catheter 102 and blood pump 101 within the patient. In that regard, securement device 132 may be any device suitable for optionally allowing and restricting movement of catheter 102 therethrough. In one aspect, the specific securement device 132 depicted in FIG. 1C is a Tuohy-Borst type device. However, any of the improved securement devices depicted and described herein may be used in place of securement device 132. In addition, although the securement devices described herein may be shown as modular units that couple to a hemostasis valve of a repositioning sheath assembly, the securement devices of the present technology may alternatively be provided as: (1) a modular unit that couples to a hemostasis valve of an introducer sheath assembly; (2) a modular unit that incorporates a hemostasis valve and couples to a hub of a repositioning sheath assembly or an introducer sheath assembly; or (3) as an integral part of a repositioning sheath assembly or an introducer sheath assembly.

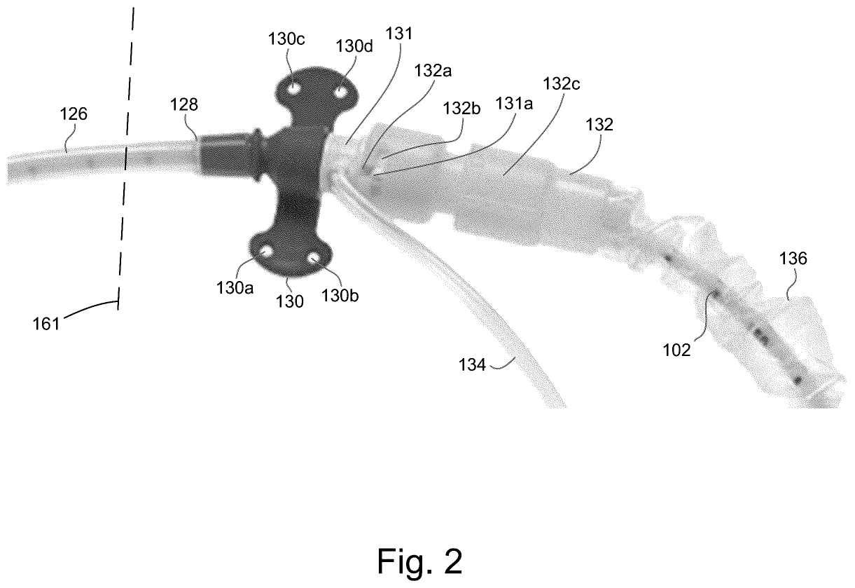

FIG. 2 shows an enlarged view of the blood pump assembly 100 and repositioning sheath assembly 180 of FIG. 1C. FIG. 2 reproduces a portion of blood pump assembly 100 and repositioning sheath assembly 180, oriented with its distal end to the left as though repositioning sheath 126 has been passed through a patient's skin surface (indicated by dashed line 161) from right to left. Oriented in this way, the proximal end 128 of repositioning sheath 126 would remain on the outside of the patient's body, and butterfly 130 may be secured to the patient's skin using sutures passed through suture eyelets 130a, 130b, 130c, and 130d.

The enlarged view of FIG. 2 shows a bayonet connection between hemostasis valve 131 and securement device 132. In that regard, the distal end of securement device 132 is configured such that it can be coupled to a proximal end of hemostasis valve 131 by pushing the two parts together and turning them relative to one another. In the example of FIG. 2, the distal portion of securement device 132 has a cylindrical collar with one or more slots, and the proximal portion of hemostasis valve 131 has a cylindrical projection with one or more pegs 131a. When the cylindrical collar of securement device 132 is advanced over the cylindrical projection of hemostasis valve 131, each peg 131a will enter one of the slots in securement device 132 at an entrance point 132a. Then, by rotating the securement device 132 relative to hemostasis valve 131, each peg 131a will move toward an end point 132b of the slot, thus preventing securement device 132 from being pulled away from hemostasis valve 131 (without first rotating the two components in the opposite direction). In addition, the internal interface between the cylindrical collar of securement device 132 and hemostasis valve 131 may include a deformable seal or gasket (e.g., a rubber washer) to both provide a seal between the parts and to provide backpressure tending to prevent peg 131a from easily moving within the slot of securement device 132. Moreover, a detent may be provided at the end points 132b of each slot in securement device 132 so that peg 131a will tend to remain in the locked state.

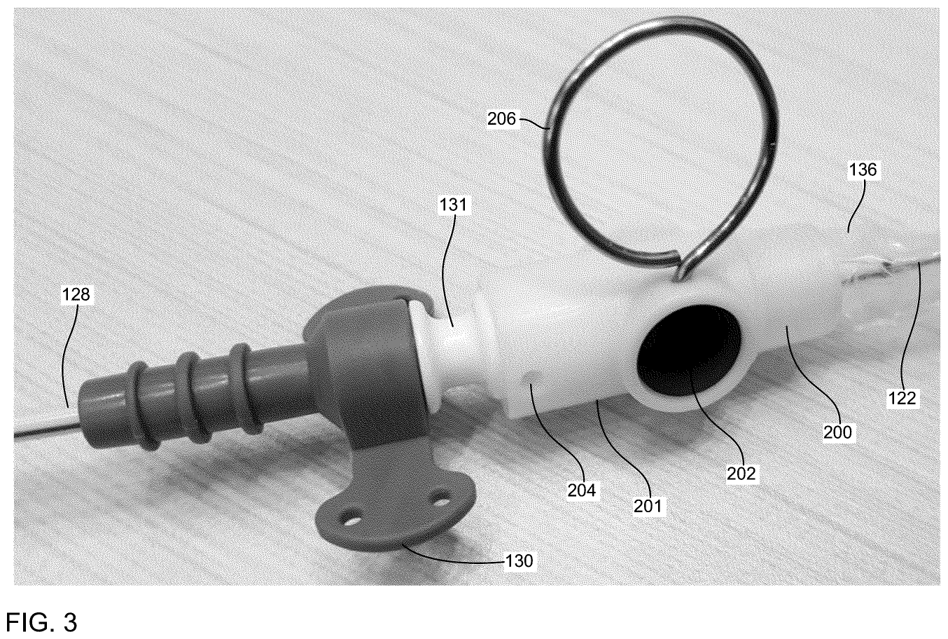

As already noted, in the example of FIGS. 1C and 2, the securement device 132 may be a Tuohy-Borst device in which a barrel 132c is rotated to vary the amount of resistance imposed on whatever object (e.g., catheter 102) is within the securement device 132. As will be discussed in further detail below, any of the securement devices depicted and described with respect to FIGS. 3-9 may be substituted into the system shown in FIGS. 1A-C and 2. Moreover, it is to be appreciated that the securement devices of the present technology may be used in association with a repositioning sheath (e.g., as a part of or connecting to a hub of the repositioning sheath) or in association with an introducer sheath (e.g., as a part of or connecting to a hub of the introducer sheath).

FIG. 3 shows an enlarged view of a blood pump and repositioning sheath assembly using an exemplary button-actuated securement device, in accordance with aspects of the disclosure. Like FIG. 2, the example of FIG. 3 shows a portion of a blood pump and repositioning sheath assembly, oriented with its distal end to the left. All shared numbers between FIG. 3 and those of FIGS. 1A-C and 2 represent the same features. Thus, the catheter 102, repositioning sheath 126 (of which only proximal end 128 is identified), butterfly 130, hemostasis valve 131, and protective sleeve 136 all are as previously described. However, unlike FIG. 2, rather than showing a conventional Tuohy-Borst type securement device, the example of FIG. 3 employs a button-actuated securement device 200.

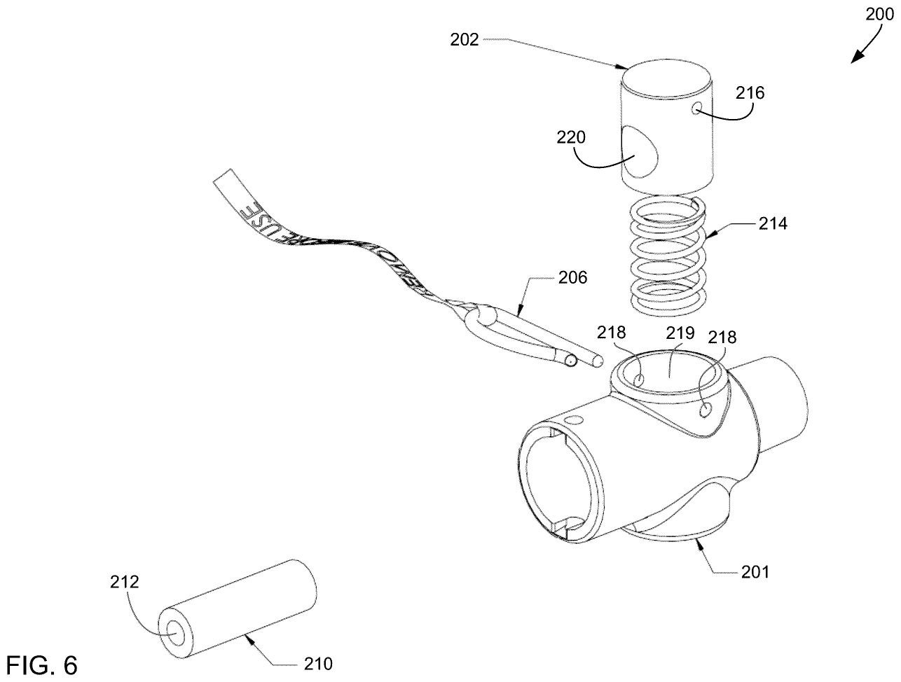

Securement device 200 comprises a housing or body 201, which houses a button 202. The body 201 of securement device 200 includes two eyelets 204 (only one of which is visible in FIG. 3), each of which are configured to engage a peg 131a (not shown, but as described above) to achieve a snap-fit between hemostasis valve 131 and securement device 200. As will be described further below with respect to FIG. 6, button 202 is spring-loaded such that it will clamp down upon an internal flexible sleeve 210 (not visible in FIG. 3), and thus provide resistance to whatever object is inserted through the lumen of that flexible sleeve whenever the button 202 is not actively being pressed. To avoid placing sustained pressure on the flexible sleeve 210 prior to use (e.g., while securement device 200 is shelved in inventory), which may cause some amount of lasting deformation of the flexible sleeve, and/or to allow easier movement of the pump at the start of a procedure, the example securement device 200 includes a retention pin 206. As will be described further below with respect to FIG. 6, retention pin 206 can be inserted through bores in body 201 and button 202 to hold button 202 in a depressed state, so that the flexible sleeve 210 will not be subjected to pressure by button 202.

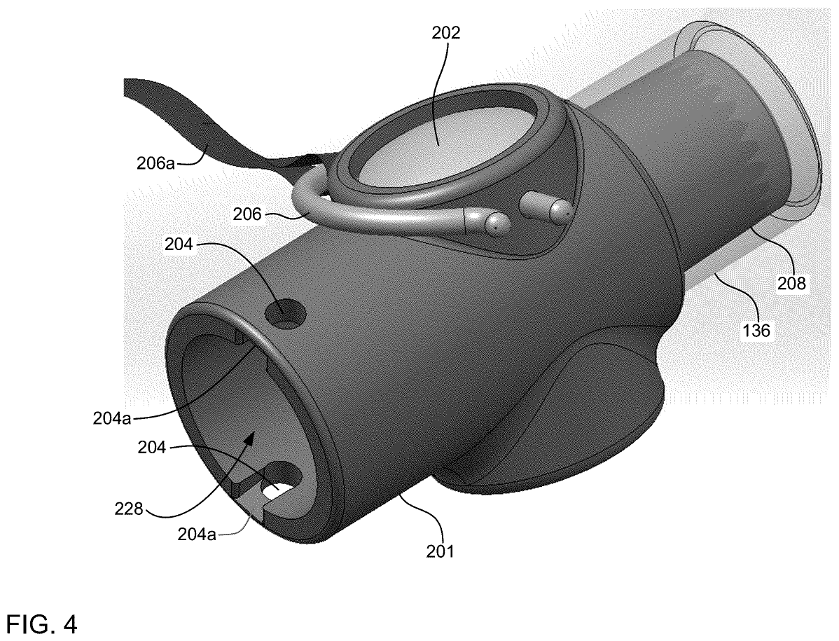

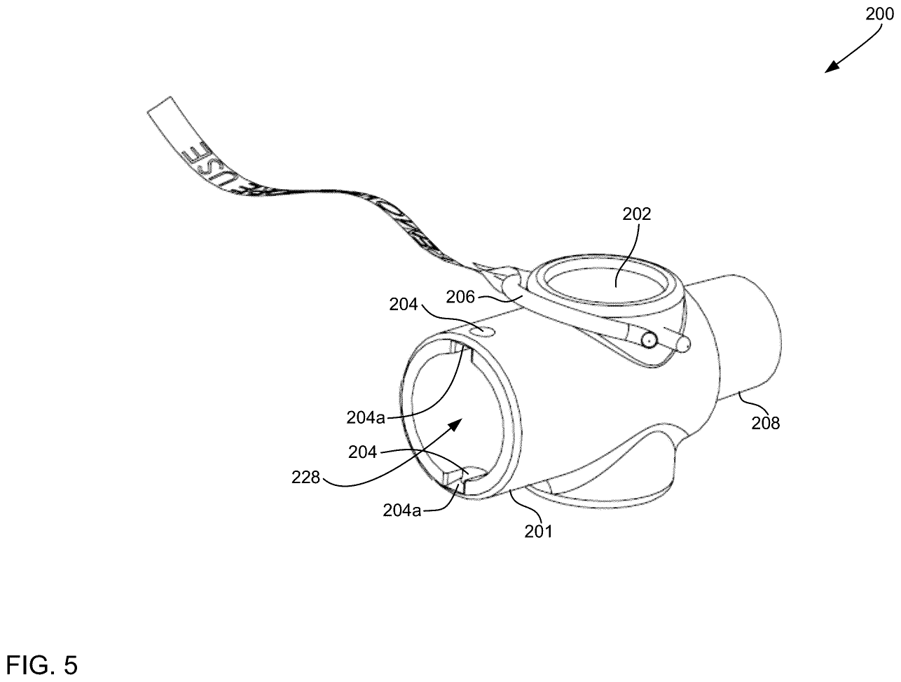

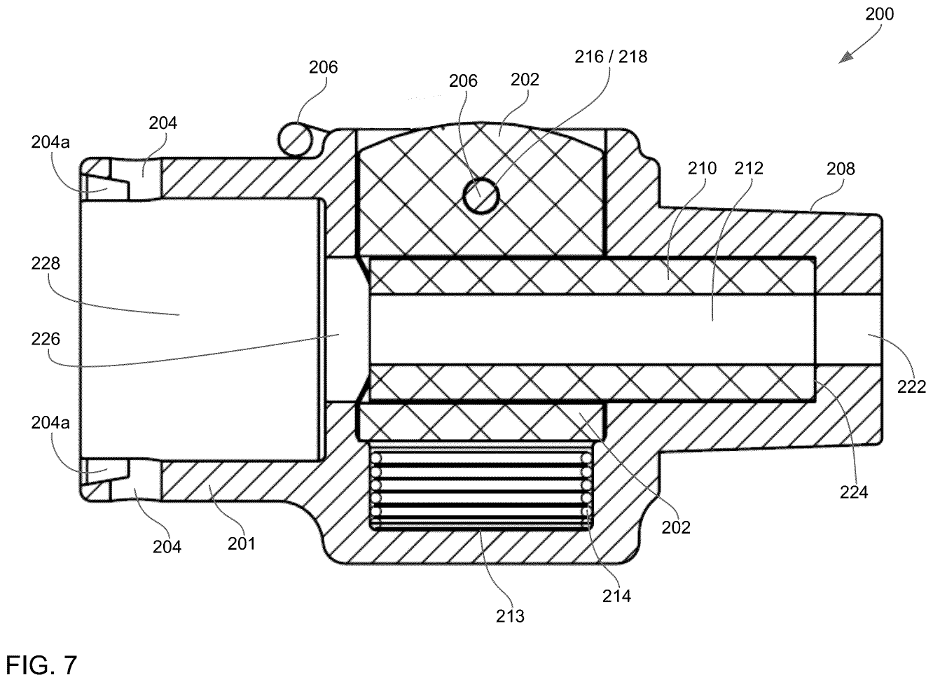

FIG. 4 shows an isometric view of a three-dimensional model of the exemplary button-actuated securing device of FIG. 3, and FIG. 5 shows a line-drawing thereof. As FIGS. 4 and 5 show the securing device 200 in isolation, the distal lumen 228 of body 201 can be seen in more detail. In particular, two slots 204a are provided which correspond to the diameter of eyelets 204. Slots 204a ramp up slightly in the proximal direction such that, when the cylindrical projection of hemostasis valve 131 is advanced into the cylindrical collar at the distal end of body 201, the resistance between pegs 131a and slots 204a will gradually increase until both pegs 131a “snap” into engagement with both eyelets 204. The ramping of slots 204a can also be seen in the cross-sectional view of FIG. 7.

The three-dimensional model of FIG. 4 also more clearly shows that body 201 has a proximal collar 208 that is configured to couple with protective sleeve 136. The coupling between proximal collar 208 and protective sleeve 136 may be accomplished by any suitable way. For example, protective sleeve 136 may be bonded to collar 208 or affixed thereto using adhesives, etc. In addition, protective sleeve 136 may be removably coupled to collar 208 using any suitable mechanical coupling, such as a ring mounted on collar 208 that can be screwed down or otherwise moved into engagement with another surface of body 201 in order to trap the end of protective sleeve 136.

The three-dimensional model and line-drawing of FIGS. 4 and 5 also show a retention pin 206 that is slightly different than what is shown in FIG. 3. As in FIG. 3, retention pin 206 is optional, and any suitable shape or configuration of retention pin may be employed.

FIG. 6 shows an exploded view of the exemplary button-actuated securing device of FIG. 3. In this exploded view, it can be seen that button 202 has two bores. Bore 216 is configured to allow passage of retention pin 206 through a portion of button 202. Body 201 has two identical or similarly sized bores 218. Accordingly, bores 216 and 218 allow retention pin 206 to be passed through both body 201 and button 202, thus holding button 202 in a depressed state.

Button 202 also has a second bore 220 that is sized to allow passage of flexible sleeve 210. Flexible sleeve 210 may be comprised of any suitable material such as silicone, etc. Flexible sleeve 210 has a lumen 212 that is sized to allow passage of whatever device is intended to be passed through securement device 200. Thus, if securement device 200 is used in the blood pump and repositioning sheath assembly such as those shown in FIGS. 1-3, lumen 212 may be sized to allow passage of catheter 102 when flexible sleeve 210 is in a relaxed, uncompressed state. The dimensions and materials used for flexible sleeve 210 may be chosen in part to provide a desired amount of friction between the lumen 212 and whatever medical device is intended to be passed through securement device 200, and/or to provide sufficient cushioning to the medical device to prevent damage to the medical device when the button 202 of the securement device 200 is not being pressed. For example, if securement device 200 is used in the blood pump and repositioning sheath assembly such as those shown in FIGS. 1-3, the flexible sleeve 210 may be configured by choosing a particular material, wall thickness, etc. to provide sufficient friction to the catheter 102 to prevent movement of the catheter 102 when the button 202 is not being pressed, while also avoiding crimping the catheter 102 and/or adversely impacting any electrical wires, purge lumens, flexible drive shafts, etc. which may pass through the catheter 102.

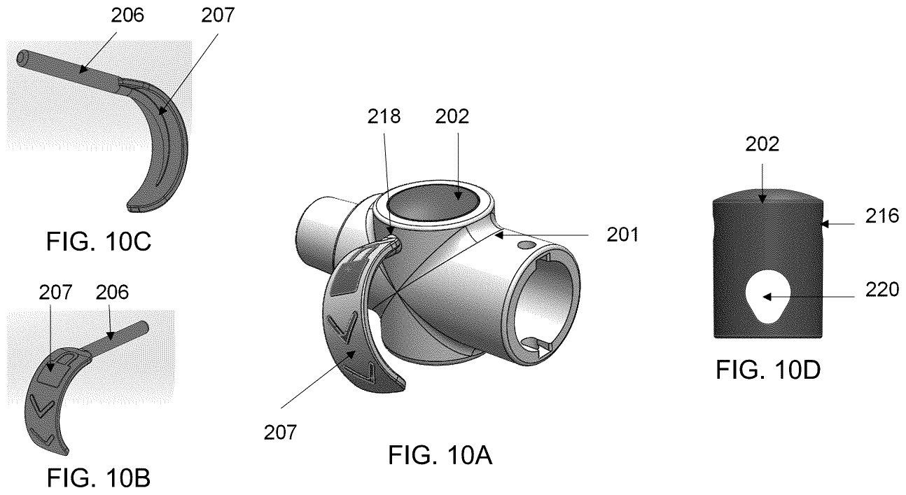

A different configuration for the retention pin is illustrated in FIGS. 10A-10D. As illustrated in FIG. 10A, FIG. 10B, and FIG. 10C, the retention pin 206 has a curved handle 207 that enables a user to remove the pin 206 simply by inserting their finger behind the handle 207 and removing the pin 206 from the bore 216 in button 202 and from the bore 218 in the body 201. As illustrated in FIG. 10D, the second bore 220 in the button 202 is tapered toward the lower portion of the button 202 received into the body 201. Therefore, the second bore 220 has a larger diameter at the top portion of the second bore 220 and a smaller diameter at the lower portion of the second bore 220, the “top” and “lower” portions of the button 202 being in relation to the “top” portion of the body 201 into which the button 202 is received. As described in detail above, this design facilitates the action of button on the catheter so that the catheter is held securely, but not crimped, when the button 202 is in the compressed (depressed) state.

As will be explained further below with respect to FIG. 7, body 201 includes a medial lumen 226 (not visible in FIG. 6) that is sized to retain flexible sleeve 210. In addition, body 201 includes a recess 213 (also not visible in FIG. 6) configured to retain spring 214 underneath button 202. Although spring 214 is depicted as a coil-spring, any suitable spring member may be substituted, such as a leaf spring, elastomeric element, living hinge, etc. When securement device 200 is assembled such that spring 214 is held within recess 213, and the flexible sleeve 210 is threaded through the bore 220 of button 202 and through the medial lumen 226 of body 201, spring 214 will be in compression, and thus providing a force tending to push button 202 upward and out of button cavity 219. This force will in turn cause bore 220 to provide a force tending to push flexible sleeve 210 upward, trapping it against the top of medial lumen 226. By selecting a spring 214 of sufficient strength, and a flexible sleeve 210 that is sufficiently flexible, this upward force on button 202 will cause flexible sleeve 210 to deform, and lumen 212 to pinch whatever object may be inserted through it (e.g., catheter 102), thus resisting movement of the object through lumen 212. Conversely, by depressing button 202, spring 214 will be further compressed, and the force on flexible sleeve 210 will be reduced or removed, allowing flexible sleeve 210 to return to a relaxed state in which lumen 212 will allow passage of whatever object is inserted through it (e.g., catheter 102). Either or both of the alternative retention pin design and the alternative second bore design described herein with respect to FIGS. 10A-10D may be combined with the other embodiments of the button-actuated securing device described herein.

FIG. 7 shows a cross-sectional profile view of the exemplary button-actuated securing device of FIG. 3. All reference numerals in common with FIGS. 3-6 identify the same features described above. As can be seen from the cross-sectional view of FIG. 7, body 201 has a proximal lumen 222 of substantially similar size to lumen 212, a medial lumen 226 of substantially similar size to the relaxed outer diameter of flexible sleeve 210, and a distal lumen 226 of substantially similar size to the cylindrical projection of hemostasis valve 131. FIG. 7 shows securement device 200 with retention pin 206 having been inserted through bores 216 and 218, and thus shows button 202 in a depressed state. As described above, once retention pin 206 is removed, spring 214 will begin to press upward on the bottom surface of button 202, which will in turn cause bore 220 of button 202 to press flexible sleeve 210 against the top of medial lumen 226, resulting in compression of the flexible sleeve 210 and its lumen 212.

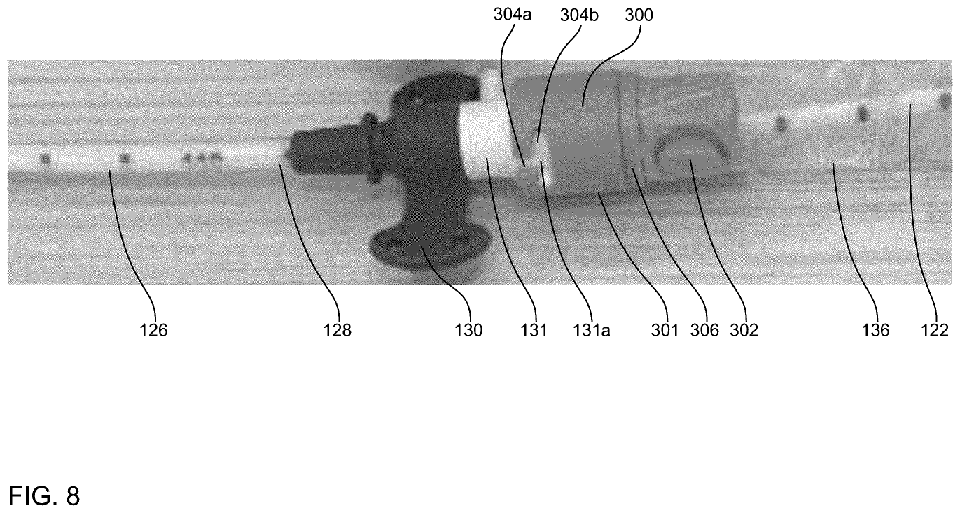

FIG. 8 shows an enlarged view of a blood pump and repositioning sheath assembly using an exemplary button-actuated securement device, in accordance with aspects of the disclosure. In that regard, like FIGS. 2 and 3, the example of FIG. 8 shows a portion of a blood pump and repositioning sheath assembly, oriented with its distal end to the left. All shared numbers between FIG. 8 and those of FIGS. 1-3 represent the same features. Thus, the catheter 102, repositioning sheath 126 (and its proximal end 128), butterfly 130, hemostasis valve 131, and protective sleeve 136 all are as previously described. Like FIGS. 3-7, FIG. 8 also shows a securement device 300 which includes a button 302, and which is configured to restrict movement of an object inserted through it in the same way as described above with respect to FIGS. 3-7. However, unlike FIGS. 3-7, the securement device 300 of FIG. 8 is configured to be coupled to hemostasis valve using a bayonet connection similar to that which is described above with respect to FIG. 2.

In that regard, in FIG. 8, the distal end of body 301 of securement device 300 is configured such that it can be coupled to a proximal end of hemostasis valve 131 by pushing the two parts together and turning them relative to one another. As with the example of FIGS. 1 and 2, the distal portion of securement device 300 has a cylindrical collar with one or more slots, and the proximal portion of hemostasis valve 131 has a cylindrical projection with one or more pegs 131a. When the cylindrical collar of securement device 300 is advanced over the cylindrical projection of hemostasis valve 131, each peg 131a will enter one of the slots in securement device 300 at an entrance point 304a. Then, by rotating the securement device 300 relative to hemostasis valve 131, each peg 131a will move toward an end point 304b of the slot, thus preventing securement device 300 from being pulled away from hemostasis valve 131 (without first rotating the two components in the opposite direction). In addition, the internal interface between the cylindrical collar of securement device 300 and hemostasis valve 131 may include a deformable seal or gasket (e.g., a rubber washer) to both provide a seal between the parts and to provide backpressure tending to prevent peg 131a from easily moving within the slot of securement device 300. Moreover, a detent may be provided at the end points 304b of each slot in securement device 300 so that peg 131a will tend to remain in the locked state.

In addition, in the example of FIG. 8, protective sleeve 136 extends over button 302 to prevent the possibility of fluids, microorganisms, etc. from entering securement device 300 around the edges of button 302, and thus contaminating catheter 102 where it passes within securement device 300. Securement device 300 has a retaining ring 306 configured to trap the end of protective sleeve 136 against an opposing surface of body 301. As described above, this trapping arrangement may be accomplished in any suitable way. Likewise, in some aspects of the technology, protective sleeve 136 may be bonded directly to ring 306 and/or body 301, or affixed thereto using an adhesive.

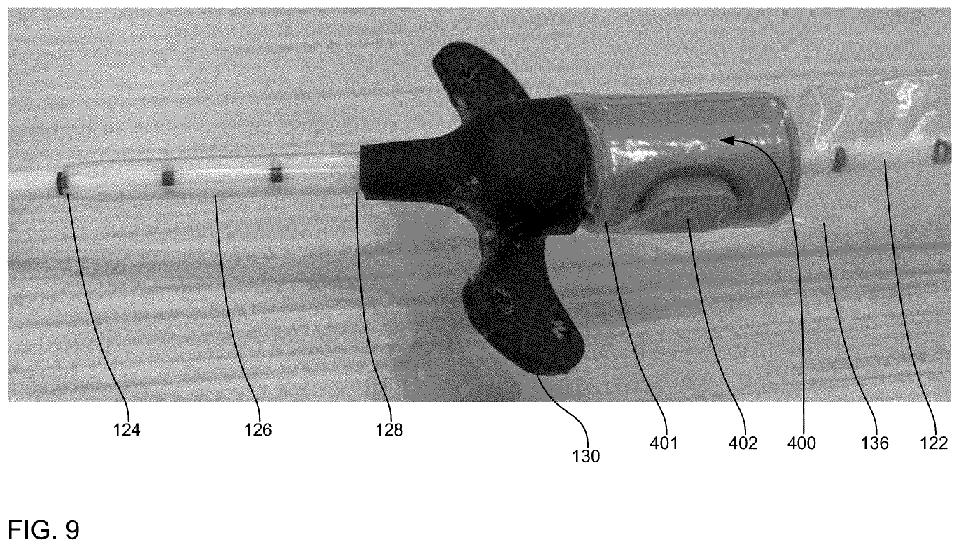

FIG. 9 shows an enlarged view of a blood pump and repositioning sheath assembly using an exemplary button-actuated securement device, in accordance with aspects of the disclosure. Here as well, the example of FIG. 8 shows a portion of a blood pump and repositioning sheath assembly, oriented with its distal end to the left. All shared numbers between FIG. 9 and those of FIGS. 1-3 represent the same features. Thus, the catheter 102, repositioning sheath 126 (and its distal end 124 and proximal end 128), butterfly 130, and protective sleeve 136 all are as previously described. FIG. 9 also shows a securement device 400 which includes a button 402, and which is configured to restrict movement of an object inserted through it in the same way as described above with respect to FIGS. 3-7. However, unlike FIGS. 1-8, the securement device 400 of FIG. 9 includes a hemostasis valve (not shown) within it. As such, the portion of the blood pump and repositioning sheath assembly shown in FIG. 9 does not require a separate hemostasis valve 131, and therefore the body 401 of securement device 400 does not require any features to enable a coupling (e.g., a bayonet connection, snap-fit connection, etc.) between it and hemostasis valve 131. In that regard, in some aspects of the technology, securement device 400 may be a modular unit that attaches directly to butterfly 130 using any suitable coupling arrangement. However, in other aspects of the technology, securement device 400 may be integral to the hub of the repositioning sheath assembly, and thus repositioning sheath 126, butterfly 130, and securement device 400 may be part of a single repositioning sheath assembly. Likewise, as mentioned above, securement device 400 may also be configured to work with or be an integral part of an introducer sheath assembly.

As described herein, the intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. Once positioned in this way, the intracardiac blood pump assembly 100 may deliver blood from the blood inflow cage 114, which sits inside the left ventricle, through cannula 110, to the blood outflow cage 106, which sits inside the ascending aorta. In some aspects of the technology, intracardiac blood pump assembly 100 may be configured such that bend 118 will rest against a predetermined portion of the patient's heart when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

While and/or after the blood pump 101 is inserted into the intended location (e.g., the left ventricle and ascending aorta in the case of a left heart pump) of the patient, the precise orientation and position of blood pump 101 may need to be adjusted to place the inlet 114 and outlet 106 in the desired positions relative to the body structures of the patient. For example, to avoid suction with the tissues and surfaces in the left ventricle, it may be desirable for the inlet 114 to be positioned in the free space of the left ventricle such that the inlet 114 does not contact the inner walls of the left ventricle or the chordae that actuate the mitral valve. This position may be achieved by orienting the distal portion 111 of the blood pump 101 toward the apex of the left ventricle and away from the chordae that actuate the mitral valve and having appropriate spacing away from the internal walls of the left ventricle.

In some instances, when the pump 101 is inserted into the patient's heart, the pump 101 may not be in the desired orientation to effectively pump blood and prevent suction events. This may occur for several reasons. For example, prior to being inserted into the patient, the catheter 102 and pump 101 may have an inherent or resting state shape. For purposes herein, the inherent or resting state shape is the shape the catheter 102 and pump 101 take when no external forces or stresses are being applied to the catheter 102 and pump 101. For example, when at rest and exterior to the patient, e.g., placed on a flat surface such as a table, the pump 101 and catheter 102 may be disposed in the same two-dimension plane (e.g., in this case, defined by the surface of the table). Due to the inherent resting state shape of the catheter 102 and pump 101, when the pump 101 is inserted into the patient's heart, the pump 101 may not be in the desired orientation or position and thus may need to be adjusted to achieve the desired position. For example, the resting state shape of the catheter 102 and pump 101 may cause the distal portion 111 of the pump, including the inlet 114, to initially be biased toward and in contact with the ventricular walls or other structures in the left ventricle, which may cause the inlet 114 to be blocked or reduce its capacity to draw in blood into the cannula 110. As will be further appreciated, due to movement of the patient and/or movement of the pump during use, the position of the pump may need to be adjusted for continued use.

In some instances, to alter the position of the pump 101 within the patient, a user may attempt to grasp the portion of the catheter 102 that is exterior to the patient and apply torque to the catheter 102 to torsion/twist the catheter 102 and thereby rotate the cannula 110 to change the orientation of distal portion 111 of pump 101 within the patient's heart. Moreover, the user may pull (proximally) or push (distally) the catheter 102 into or out of the patient. Referring to FIG. 1C, the portion of the catheter 102 that is exterior to the patient when the pump 101 is inserted in the patient is indicated by reference numeral 181. Portion 181 is the portion of catheter 102 that is distal of plug 138 and proximal of fixation device or butterfly 130 when butterfly 130 is affixed to the patient proximate to the access site. Some or all of portion 181 may be covered by protection sleeve 136 and/or a securement device, such as one of securement devices 132, 200, 300, 400.

However, the user may find that portion 181 of catheter 102 or any other portion of assembly 100 exterior to the patient difficult to sufficiently grip and manually manipulate the catheter 102 in order to manually steer the blood pump 101.

As described herein, the inventors have recognized the benefits of improving a user's ability to apply torque to a catheter of a catheter assembly and adjust a position of the blood pump at the distal end of the catheter. The present technology provides devices and assemblies with features that enhance the user's ability to grip the portions of the pump assembly (such as portion 181 of catheter 102) that are exterior to the patient to give the user sufficient control to steer pump 101 by torquing, pushing, or pulling the catheter 102.

In some aspects of the present technology, a securement device, such as any of securement devices 132, 200, 300, 400 described herein, may be configured to enable the user to grip portion 181 of the catheter 102 with sufficient friction, force, and/or stability to torque, push, and/or pull catheter 102 and as needed by a clinician to steer blood pump 101 to a desired position within the patient.

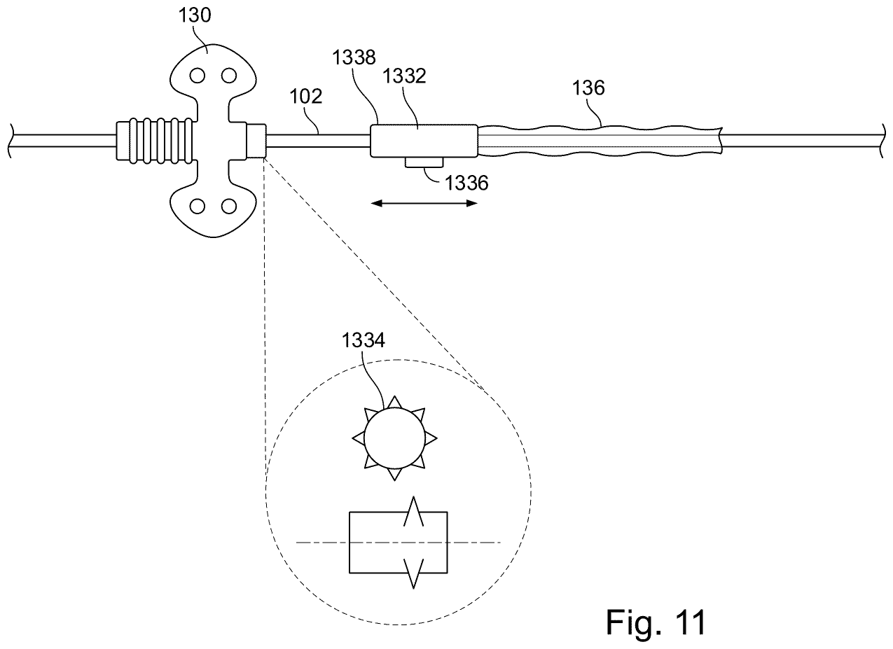

For example, as shown in FIG. 11, a securement device 1332 is provided in accordance with one aspect or embodiment of the present technology. It is to be appreciated that securement device 1332 may include any of the features of securement devices 132, 200, 300, 400 described herein. In this regard, the securement device 1332 may include a securing mechanism configured to be selectively engaged (e.g., by pressing a button or turning a barrel) to restrict or permit the movement of an object (e.g., catheter 102) inserted through the securement device 1332. For example, the securing mechanism may comprise a Tuohy-Borst device with a rotatable barrel (such as barrel 132c) as used in securement device 132 to vary the amount of resistance imposed on the catheter 102 inserted through the securement device. Alternatively, the securing mechanism may comprise a spring-loaded button 202, 302, 402 as used in securement devices 200, 300, 400 described above that compresses a flexible sleeve (such as sleeve 210) to resist the movement of the catheter 102 through the securement device.

When the securing mechanism of securement device 1332 is not engaged (and thus not restricting the movement of catheter 102), the securement device 1332 may be configured to be slidable or otherwise displaceable over catheter 102 (e.g., along portion 181). In this regard, the securing device may be permanently attached to the catheter (e.g., at portion 181). In other embodiments, the securing device may be an accessory that is attachable to the catheter (e.g., at portion 181) to allow a clinician to move the catheter and, thus, pump, into a desired location and then remove the securing mechanism when not in use.

In some embodiments, the length of securement device 1332 from its proximal to distal end may be selected to enable the user to easily grip the exterior of securement device 1332 in a stable manner. For example, in some embodiments, the length of the device 1332 may be between 1 and 6 inches, although it will appreciated by the skilled person that other suitable ranges may be selected depending upon the configuration of the device.

Securement device 1332 may include a button, slider, thumbwheel, collar or other actuating member 1336 for actuating the securing mechanism of securement device 1332 to internally grip the exterior of catheter 102. In some embodiments, the device may also include a separate gripping mechanism for gripping catheter 102 that is separate from the securing mechanism (i.e., spring-loaded button), such as that described above. The actuating member 1336 may be selectively engaged by the user to cause the securement device 1332 to grip the exterior of the catheter 102 inserted through device 1332. With the catheter 102 gripped by securement device 1332, the user may grasp the exterior of securement device 1332 and push, pull, and/or torque the securement device 1332 to thereby push, pull, or torque the catheter 102 to position the blood pump 101 appropriately within the patient. In one aspect, when actuating member 1336 is engaged by the user, an inner diameter of a component of the securing mechanism of securement device 1332 is reduced to grip the exterior of catheter 102.

In one aspect, the proximal end of the butterfly 130 may include an attachment member 1334 (e.g., a magnet or mechanical coupling member) that engages and attaches to a corresponding attachment member 1338 (e.g., a magnet or mechanical coupling member) at the distal end of securement device 1332. In this way, the butterfly 130 may be securely coupled to securement device 1332. Attachment members 1334, 1338 may be configured to automatically attach to each other when manually brought together by a user (e.g., by way of a snap fit connection, magnetic connection, or any other suitable connection). In some aspects, securement device 1332 may include a second actuating member (not shown) accessible on the exterior of securement device 1332. The second actuating member may be configured to change between different states or positions. For example, in some embodiments, a first state or position may cause members 1334, 1338 to attach when members 1334, 1338 are brought into contact with each other. A second state or position may cause members 1334, 1338 to detach from each other. The second actuating member may be a button, knob, slider, etc., or other means to control the attachment or detachment of members 1334, 1338.

In one aspect, the securement device 1332 may be coupled to the sleeve 136. For example, as shown in FIG. 11, the proximal end of securement device 1332 may be coupled to the distal end (or a distal portion) of sleeve 136. In some aspects, securement device 1332 may be coupled, attached, or otherwise joined (e.g., using adhesives, retaining rings, or other suitable techniques) to the sleeve 136.

In one aspect, actuating member 1336 may be actuated by a user to achieve a locked state. In the locked sate, without further user engagement (e.g., without the user applying continued force to engage actuating member 1336), the securement device 1332 may continue gripping catheter 102 until the actuating member 1336 is actuated by a user to achieve an unlocked state. As will be appreciated, in embodiments in which the actuating member is fixedly attached to the catheter, it may remain connected to the catheter whether or not in the locked state.

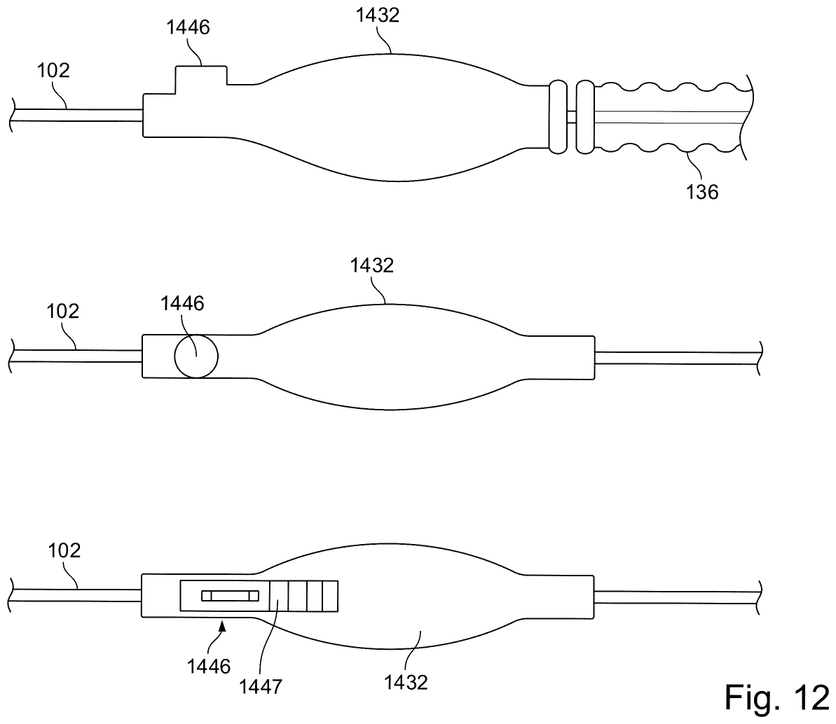

Referring to FIG. 12, another securement device 1432 that is configured to grip the exterior of catheter 102 to permit the user to push, pull, and/or torque the catheter 102 is illustrated in accordance with the present technology. It is to be appreciated that securement device 1432 may include any of the features of securement devices 132, 200, 300, 400 described herein. In this regard, the securement device 1432 may include a securing mechanism configured to be selectively engaged to restrict or permit the movement of an object (e.g., catheter 102) inserted through the securement device 1432. For example, the securing mechanism may comprise a Tuohy-Borst device with a rotatable barrel (such as barrel 132c) as used in securement device 132 to vary the amount of resistance imposed on the catheter 102 inserted through the securement device. Alternatively, the securing mechanism may comprise a spring-loaded button 202, 302, 402 as used in securement devices 200, 300, 400 described above that compresses a flexible sleeve 210 to resist the movement of the catheter 102 through the securement device.

In any case, securement device 1432 may have a housing with an exterior surface that has an enlarged ergonomic shape and a length (measured from proximal to distal) that permits a user to easily grip the exterior of securement device 1432 and manually push, pull, and/or torque securement device 1432 with a single hand. As shown, the ergonomic shape of the exterior may include a convex surface. In one aspect, securement device 1432 includes a button, slider, knob, or other actuating member 1446 to actuate the securing mechanism of securement device 1432 to internally grip the exterior of catheter 102. As with other embodiments, the securing device 1432 also may include a gripping mechanism for gripping catheter 102 that is separate from the securing mechanism (e.g., spring-loaded button). The actuating member 1446 may be selectively engaged by the user to cause the securement device 1432 to grip the exterior of the catheter 102 inserted through device 1432. With the catheter 102 gripped by securement device 1432, the user may grasp the exterior of securement device 1432 and push, pull, or torque the securement device 1432 to thereby push, pull, or torque the catheter 102 to position the blood pump 101 appropriately within the patient. In one aspect, when actuating member 1446 is engaged by the user, an inner diameter of a component of the securing mechanism of securement device 1432 may be reduced to grip the exterior of catheter 102. In one aspect, the proximal end of securement device 1432 may be configured to be removably coupled or connected with sleeve 136. In one aspect, the securement device 1432 may be integrated with the repositioning unit 180.

In one aspect, actuating member 1446 comprises a lockable slide 1447. In a first position of the slide 1447, the securement device 1432 does not grip the exterior of the catheter 102. Instead, the actuating member 1446 may be moveable relative to the catheter when the slide is in the first position. In some embodiments, the lockable slide 1447 may be configured to lock when pushed to a second position of the slide 1447. In some embodiments, the slide is configured such that a user may maintain contact with the slide to keep the slide in the locked position. In other embodiments, the lockable slide may auto-lock with a low application of force from the user. When the slide 1447 is auto-locked, user contact with the slide 1447 can be discontinued and the slide 1447 may stay in the second position. In the second position, the slide 1447 causes the securement device 1442 to continuously grip the exterior of catheter 102 unless the user pushes slide 1447 back to the first position. If the user applies additional force to slide 1447, which causes slide 1447 to move to a third position, the securement device 1432 may be configured to apply an increased force (relative to the force applied in the second position) to the exterior of catheter 102, which allows the user to apply greater torque, pushing, or pulling force to catheter 102 through device 1442. In one aspect, advancing the slide 1447 to the opposite end of the slot or track causes the securement device 1432 to detach from butterfly 130.

It is to be appreciated that, in some aspects, securement devices 1332, 1432 may be configured to include a mechanism for applying torque to catheter 102 once gripped by securement devices 1332, 1432. For example, securement devices 1332, 1432 may include a thumbwheel, gear, knob, or other type of mechanism that is accessible to the user on an exterior surface of devices 1332, 1432 and that the user can actuate. The mechanism may be configured to transfer the torque applied by the user to catheter 102, thereby torquing catheter 102 and steering the pump 101.

In some aspects of the present technology, a securement device, such as any of securement devices 132, 200, 300, 400 may be provided with a grippable handle configured to enable the user to grip portion 181 of the catheter 102 with sufficient friction, force, and stability to torque, push, and/or pull catheter 102 accurately so that the user may steer blood pump 101 as desired within the patient.

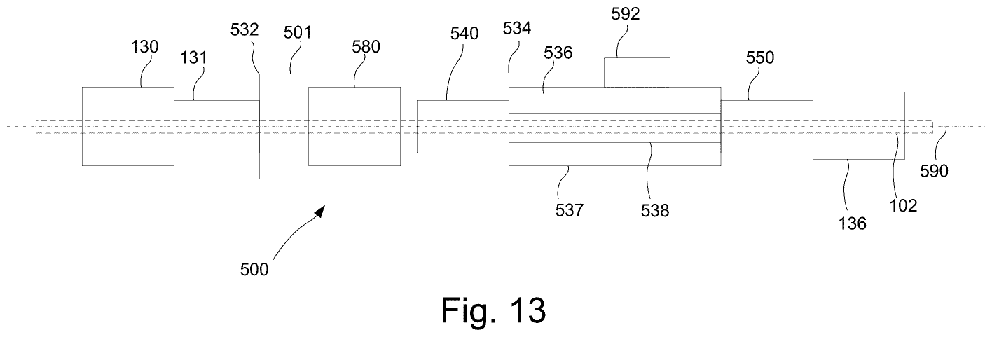

For example, FIG. 13 illustrates a securement device 500 provided with a grippable handle 536 in accordance with the present technology. Securement device 500 may include any of the features described above in relation to securement devices 132, 200, 300, 400.

In one aspect, the securement device 500 includes a housing 501 including a distal end 532 and a proximal end 534. The housing 501 may extend along a longitudinal axis 590 from the proximal end 534 to the distal end 532. The distal end 532 of the securement device 500 may be configured to couple with a fixation device 130 (e.g., a butterfly fixation device) that is attachable to the skin of the patient proximate to the access site for the assembly 100. In some aspects, distal end 532 includes a cavity configured to receive a proximal end of a hemostasis valve 131 that couples the securement device 500 to the fixation device 130. In some aspects, the fixation device 130 or the securement device 500 may be integrated with the hemostasis valve 131 and the distal end 532 of the securement device 500 couples directly with the fixation device 130.

The securement device 500 may further include a securing mechanism 580 such as any of the securing mechanisms described herein. The securing mechanism 580 may be configured to be selectively engaged by the user to restrict or permit the movement of an object (e.g., catheter 102) inserted through housing 501 along axis 590. For example, the securing mechanism 580 may comprise a Tuohy-Borst device with a rotatable barrel (such as barrel 132c) as used in securement device 132 to vary the amount of resistance imposed on the catheter 102 inserted through the securement device 500. Alternatively, the securing mechanism 580 may comprise a spring-loaded button 202, 302, 402 as used in securement devices 200, 300, 400 described above that compresses a flexible sleeve 210 to resist the movement of the catheter 102 through the securement device 500.

The securement device 500 further includes a grippable handle 536. The grippable handle 536 includes a proximal end, a distal end, an exterior surface 537, and an interior surface defining an interior lumen 538. The grippable handle 536 and the interior lumen 538 each may extend from their respective proximal to distal ends along the longitudinal axis 590. The interior lumen 538 may be configured to receive a portion of the catheter 102 that is exterior to the patient, e.g., portion 181, described above. In some aspects, the diameter of lumen 538 may be configured to be larger than the outer diameter of catheter 102 when the grippable handle 536 is in an unstressed state and free of externally applied forces. Thus, when the securing mechanism 580 is not engaged to restrict the movement of catheter 102 through housing 501 and distal end 532 is not coupled to fixation device 130, the securement device 500 is slidable over catheter 102 along the longitudinal axis 590.

In some embodiments, the distal end of the grippable handle 536 may be rotatably coupled to the proximal end 534 of housing 501 such that grippable handle 536 is rotatable relative to housing 501. The grippable handle 536 may be configured to be deformable in response to a compression force applied to the grippable handle 536. In some aspects, the grippable handle 536 may made of rubber, silicone, or another suitable material that is deformable. The compression force may be applied by a user gripping the exterior surface 537 of handle 536 while the user is applying the torque (or other movement) to move the catheter and, thus, the pump to the desired location in the patient's body. Alternatively, in some aspects, securement device 500 may include an actuator 592 (e.g., a slider, knob, thumbwheel, or other actuator) configured to apply a compression force to grippable handle 536 when actuated by a user. The actuator 592 may be configured to be lockable (e.g., as described above in relation to slide 1447) to maintain the compression force on grippable handle 536 until the actuator 592 is unlocked by the user.

When the grippable handle 536 is deformed, at least a portion of the internal lumen 538 of grippable handle 536 may deform such that the interior surface forming the lumen 538 grips the exterior of catheter 102. In this way, when the compression force is applied to grippable handle 536 and the catheter 102 is gripped, the user may advantageously torque the grippable handle 536, which transfers the torque to the gripped portion of the catheter 102 to permit the user to position the blood pump 101 appropriately within the patient. In some aspects, the exterior surface 537 of grippable handle 536 may have an ergonomic shape to aid in the user's ability to comfortably grip and apply compression to the grippable handle 536. The grippable handle 536 has a sufficient length from its proximal end to its distal end to permit a stable grasping of grippable handle 536 and the catheter 102. For example, in some embodiments, the handle may be between 1 and 6 inches long, although the handle may have other suitable lengths.

In some aspects, the grippable handle 536 is configured such that, after the compression force is removed (i.e., is no longer being applied by the user) to grippable handle 536, the grippable handle 536 (and the interior lumen 538) substantially returns to its original unstressed state.

In some aspects, securement device 500 may include a coupling mechanism 540 configured to rotatably couple the grippable handle 536 to the housing 501. In some aspects, the coupling mechanism 540 is further configured to permit the grippable handle 536 to be translated along the longitudinal axis 590 (proximally or distally) relative to housing 501. Thus, when the compression force is applied to grippable handle 536 and the catheter 102 is gripped, the user may advantageously push or pull the grippable handle 536, which transfers the push or pull force to the gripped portion of the catheter 102 to permit the user to position the blood pump 101 appropriately within the patient. In this way, securement device 500 permits the user to simultaneously rotate and longitudinally translate catheter 102 using handle 536.

In some aspects, securement device 500 may include a second coupling mechanism 550 configured to rotatably couple the grippable handle 536 to the protective sleeve 136 such that the grippable handle 536 is rotatable relative to the protective sleeve 136. In this way, the protective sleeve 136 is not torqued when the grippable handle 536 is torqued. In some embodiments, this may prevent sleeve 136 from being damaged by the rotation of handle 536.

As discussed herein, the features of securement device 500 that aid in gripping and positioning the catheter 102 may be implemented with any of the securement devices described herein.

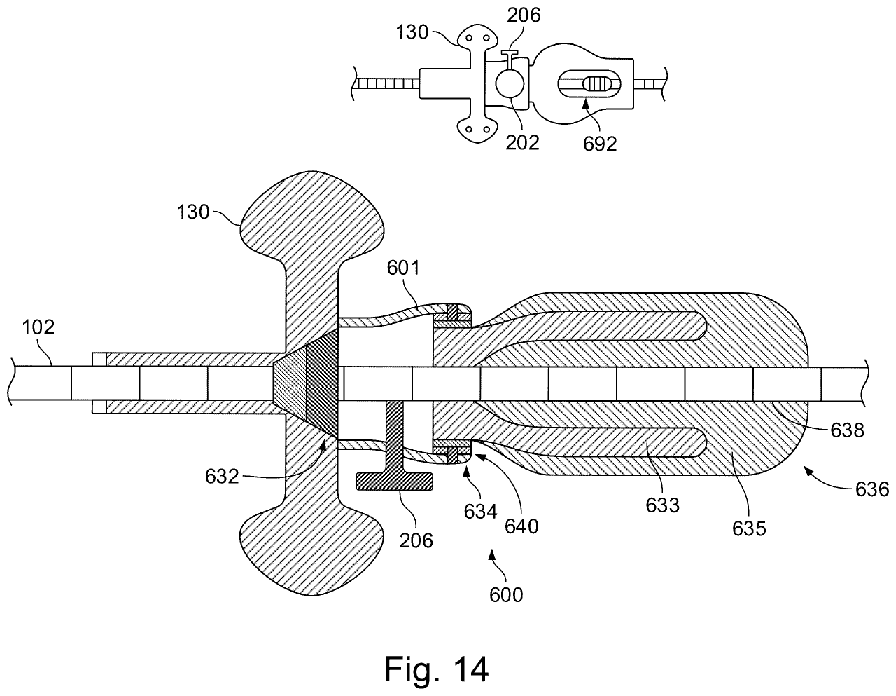

For example, referring to FIG. 14, a securement device 600 that may include a grippable handle 636 is shown in accordance with the present technology. Securement device 600 may include a housing 601 with a distal end 632 and a proximal end 634. The housing 601 may be configured to use the securing mechanism described above in relation to securement device 200 for selectively restricting the movement of catheter 102 when inserted through housing 601. In this regard, in the same manner described in relation to securement device 200, securement device 600 may include a flexible sleeve 210 (not shown), button 202, spring 214 (not shown), and retention pin 206 for restricting the movement of catheter 102. However, in the aspect shown in FIG. 14, the proximal end 634 of housing 601 may configured to be coupled with a grippable handle 636 such that grippable handle 636 is rotatable relative to housing 601. In one aspect, handle 636 may be rotatably coupled to housing 601 via a coupling mechanism 640. The coupling mechanism 640 may include bearings configured to permit handle 638 to rotate relative to housing 601.

The distal end 632 of housing 601 may be configured to couple with a fixation device 130. In some aspects, the distal end 632 may include a cavity configured to receive a proximal portion of hemostasis valve 131 and to couple the housing 601 with fixation device 130.

Handle 636 may include any of the features of handle 536 described above. In this regard, handle 636 may include an interior lumen 638 configured to receive a portion of catheter 102. Moreover, handle 638 may be deformable in response to a compression force applied to handle 638. The compression force may be applied manually by a user gripping the exterior surface of handle 638 and squeezing. Alternatively, the compression force may be applied using an actuator 692 (e.g., a button, slider, thumbwheel, etc.) configured to permit the user to selectively apply compression force to handle 638. In one aspect, the actuator 692 is a slider. The slider may be configured to be biased to a first position (e.g., by a spring) such that in the first position the slider does not apply any compression force to handle 636. The slider may be advanced to a second position such that in the second position the slider applies compression force to handle 636 to deform handle 638 such that the interior surface of handle 636 defining lumen 638 grips the catheter 102. The slider may be configured to permit a user to lock the slider in the second position such that the user's hand is free to rotate handle 636 to apply torque to catheter 102.

In one aspect, handle 636 comprises a first portion 635 and a second portion 633. The second portion 633 has a higher material hardness and is less deformable than the first portion 635. The hardness of the second portion 633 is sufficiently high to aid handle 636 in maintaining its structural integrity and to provide secure coupling to housing 601 via coupling mechanism 640. The first portion 635 is configured to be soft enough to deform in response to a compression force applied to the portion 635 to grip catheter 102 so that when a user torques the handle 636, the torque is transferred and applied to catheter 102 to steer and position pump 101.

As will be appreciated, the housing portion that engages with the handle and connects to the butterfly and the handle (e.g., a housing collar) may have any suitable cross-sectional shape. For example, the housing portion may be cylindrical, triangular, polygonal or other suitable shape.

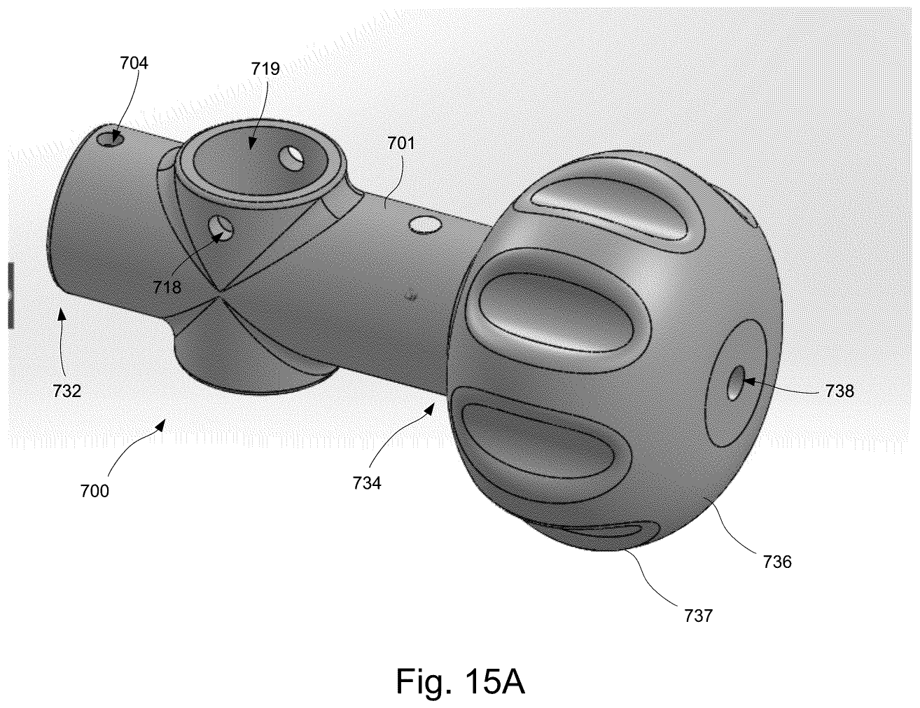

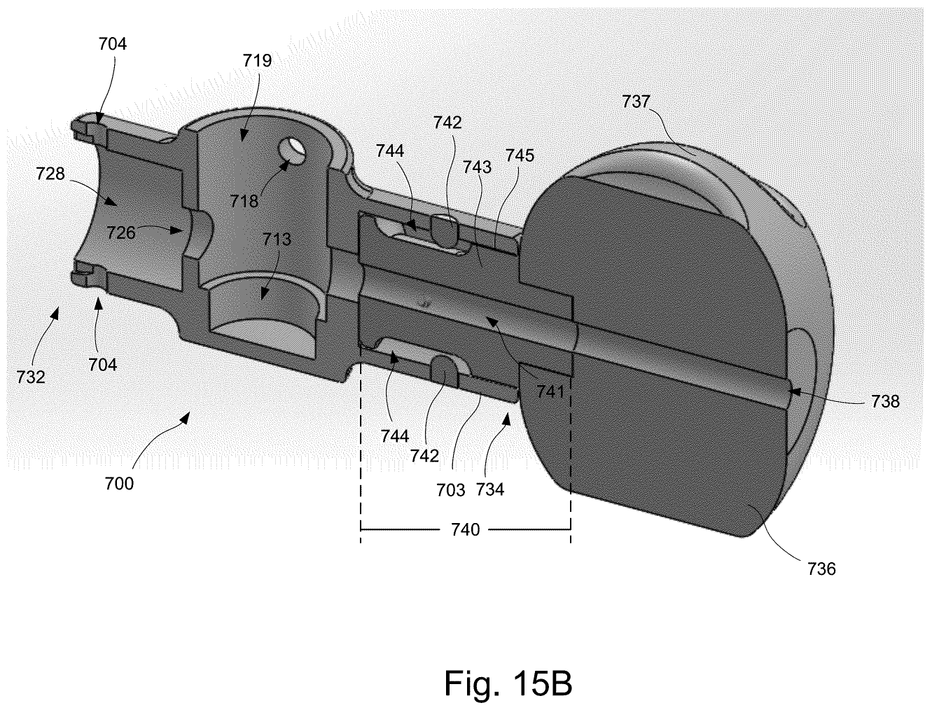

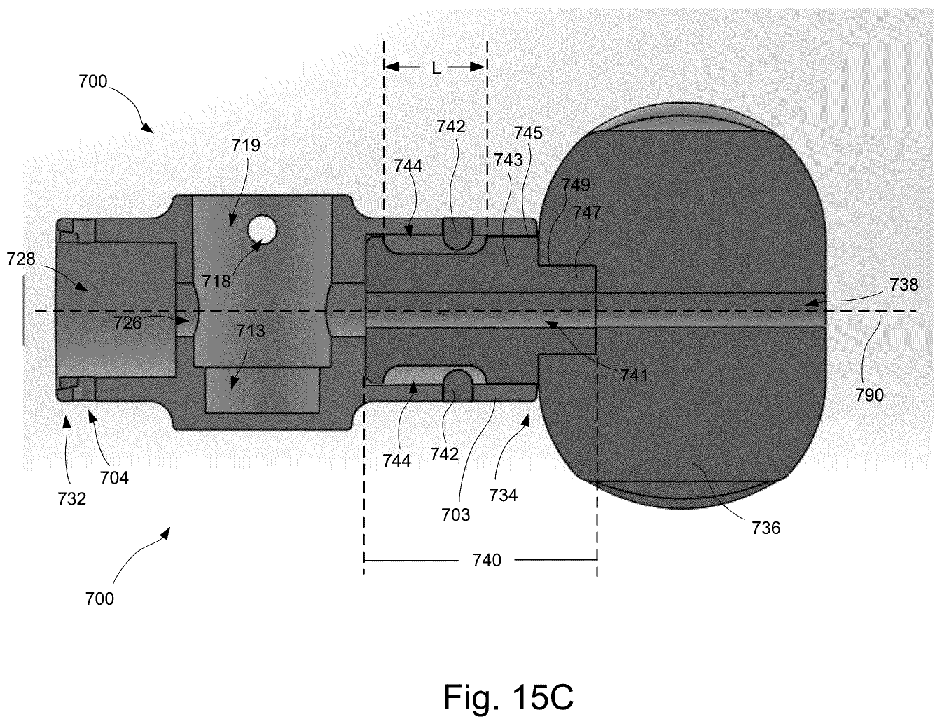

Referring to FIGS. 15A-15C, a securement device 700 provided with a grippable handle 736 is illustrated in accordance with another embodiment of the present technology. Securement device 700 may include a housing 701, a grippable handle 736, and a coupling mechanism 740. The housing 701 may include a distal end 732 and a proximal end 734. The distal end 732 may be configured to couple with a fixation device, such as fixation device 130. In some aspects, distal end 732 couples with fixation device 130 via a hemostasis valve 131. As will be described in greater detail, the coupling mechanism 740 rotatably couples the distal end of grippable handle 736 to the proximal end 734 of housing 701 such that handle 736 is rotatable relative to housing 701. The housing 701, coupling mechanism 740, and handle 736 each extend along a longitudinal axis 790 (shown in FIG. 15C). The longitudinal axis 790 extends proximally and distally.

Securement device 700 may include any of the features described herein in relation to securement device 200. It is to be appreciated that reference numbers 704, 713, 718, 719, 726, 728, 732 have the same features described herein in relation to references numbers 204, 213, 218, 219, 226, 228, 232. Thus, housing 701 is configured to be used with the button 202, spring 214, flexible sleeve 210, and retention pin 206 to selectively restrict or permit movement of the catheter 102 through the housing 701 in the same manner described herein in relation to securement device 200.

In one aspect, the coupling mechanism 740 includes a substantially cylindrical component 743 and one or more pins 742. The proximal end 734 of the housing 701 includes a collar 703 including a cylindrical cavity 745 configured to receive at least a portion of cylindrical component 743. The cylindrical component 743 is translatable along the longitudinal axis 790 relative to housing 701 and rotatable about axis 790 relative to housing 701 within the cavity 745. The cylindrical component 743 includes a slot 744 formed on the outer cylindrical circumference of the cylindrical component 743. The pins 742 extend from an interior surface of collar 703 into the slot 744.

The proximal end of cylindrical component 743 is attached to the distal end of handle 736 such that when a user pushes or pulls handle 736 to translate handle 736 along axis 790, the cylindrical component 743 is also translated along axis 790 into or out of the cavity 745 in collar 703. Moreover, component 743 and handle 736 are attached such that when a user rotates handle 736 (clockwise or counterclockwise) about axis 790, the cylindrical component 743 also rotates about axis 790. In one aspect, the cylindrical component 743 may include a proximal projection 747 and the distal end of the handle 736 may include a cavity 749 configured to receive the projection 747. The projection 747 may be inserted into the cavity 749 and attached to the surfaces forming cavity 749 to attach component 743 to handle 736.

As shown in FIG. 15C, the slot 744 has a length L from a distal end of the slot 744 to a proximal end of the slot 744. As handle 736 is translated along axis 790, the pins 742 abut against the distal and proximal ends of the slot 744 to define the length L of translation that the coupling mechanism 740 permits the handle 736 and cylindrical component 743 to travel along axis 790. In one aspect, the length L is in a range of 0.5 to 4 centimeters (cm). In another aspect, the length L is in a range of 1 to 2 cm. It will be appreciated that other suitable lengths may be chosen. In one aspect, as shown in FIG. 15C, when the handle 736 is fully translated in the distal direction along axis 790, the distal end of the cylindrical component abuts the housing 701 at the distal end of cavity 745. As will be appreciated, in such embodiments, the user may translate the position of the pump (e.g., back and forth) at the same time or at different times to facilitate rotation and proper positioning of the pump.

In one aspect, the slot 744 extends around the entire circumference of cylindrical component 743. In this aspect, coupling mechanism 740 permits handle 736 to be rotated about axis 790 (in either direction) by any amount desired by the user (e.g., 90 degrees, 180 degrees, 360 degrees, 720 degrees etc.) In another aspect, slot 744 may be configured to limit the amount that handle 736 may be rotated to limit the amount of torque that may be placed on catheter 102. For example, the slot 744 may be configured to only extend around the circumference of cylindrical component 743 by a predetermined number of degrees rotation (e.g., 90 degrees, 120 degrees, 180 degrees, etc.) such that the pin 742 will abut against the sides of the slot to restrict rotation of handle 736 within defined rotational limits. Alternatively, slot 744 may include one or more dividers (not shown) extending longitudinally in slot 744. The pins 742 may abut against these dividers to restrict the rotation of handle 736 within defined rotational limits.

The handle 736 may include an interior lumen 738, which extends along the longitudinal axis 790 and is configured to receive a portion of catheter 102. As described herein, the lumen 738 may have a diameter that is larger than outer diameter of catheter 102. The cylindrical component 743 includes an interior lumen 741, which extends along the longitudinal axis 790 and is configured to receive at least a portion of catheter 102. In one aspect, when handle 736 is not compressed and not deformed, the lumen 738 may have a diameter that is the same as the diameter of lumen 741. Lumens 738 and 741 may be disposed adjacent to each other to form a longer lumen extending from the proximal end of handle 736 to the distal end of component 743.

As described in relation to handles 536 and 636, handle 736 also may be deformable in response to a compression force applied to the handle 736 to thereby deform lumen 738. The compression force may be applied by a user gripping the exterior surface 737 of handle 736 and squeezing the handle 736. Alternatively, as described herein, handle 736 may include an actuator that may be actuated by the user to apply the compression force to handle 736.

The catheter 102 may be inserted through lumens 738 and 741 and through the securing mechanism 719 that may include securing structures described elsewhere herein (e.g., button 202, sleeve 210, etc.) of device 700. Then, a user may compress handle 736 to deform lumen 738 such that the interior surface of handle 736 that forms lumen 738 grips the exterior of the portion of catheter 102 disposed in lumen 738. With the handle 736 gripping the catheter 102 and the securing mechanism of device 700 not restricting the movement of catheter 102, the user may torque handle 736 to rotate handle 736. The torque applied to handle 736 may then be transferred to catheter 102 to rotate catheter 102. Moreover, with the handle 736 gripping the catheter 102 and the securing mechanism 719 of device 700 not restricting the movement of catheter 102, the user may translate the handle 736 along the longitudinal axis 790 by pushing or pulling handle 736 proximally or distally. The translation of handle 736 is transferred to catheter 102 to push or pull catheter 102 along axis 790. It is to be appreciated that securing device 700 is configured such that the user is permitted to rotate and longitudinally translate catheter 102 simultaneously (or at separate times) using handle 736. In this way, handle 736 may be used to torque, push, and/or pull the catheter 102 to steer pump 101. The handle 736 may be made of a silicone, rubber, or other suitable material that is deformable and configured to grip catheter 102 when compressed. The handle 736 may be configured to return to its original state/shape (prior to compression) when the compression force is removed from handle 736.

As shown, the handle 736 may be of a sufficient size and length and may have a shape which aids the user to grip and rotate handle 736. As best seen in FIG. 15A, the exterior surface 737 of handle 736 may include one or more concave and/or convex portions that aid the user to grip and apply torque to handle 736.

It is to be appreciated that, in some aspects, when the securing mechanism of device 700 (e.g., including button 202) is not engaged to restrict movement of catheter 102 and the handle 736 is not compressed, the securing device 700 and the catheter 102 are displaceable relative to each other on axis 790. In this aspect the diameters of lumens 738, 741 and the lumen of flexible sleeve 210 are larger than the outer diameter of catheter 102.

As described above in relation to FIG. 13, the securement device 700 may be provided with a second coupling mechanism configured to rotatably couple the proximal end of the grippable handle 736 to the protective sleeve 136.

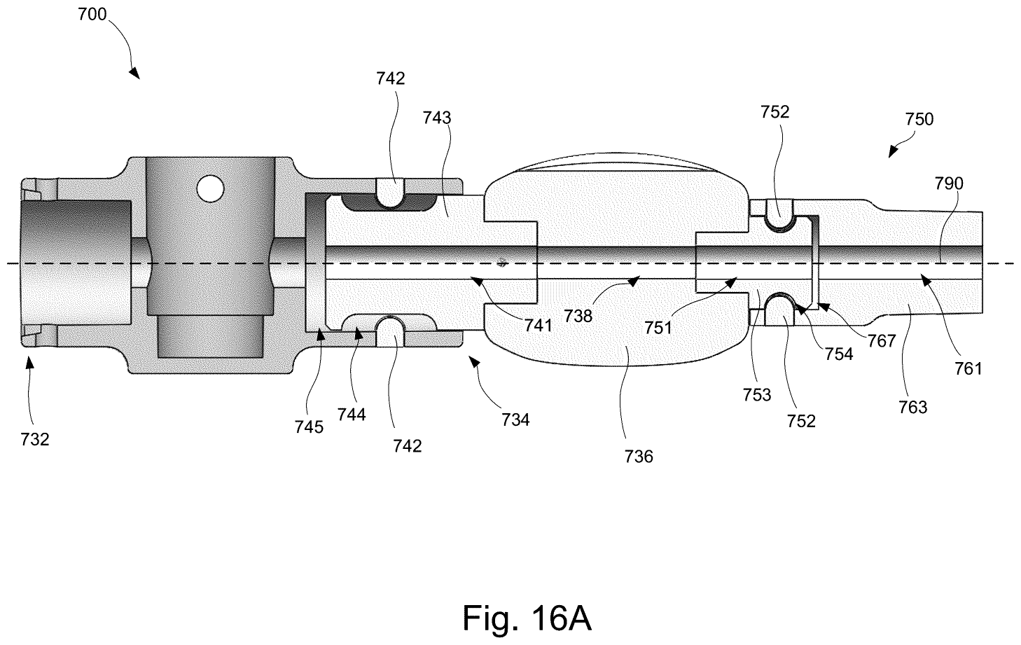

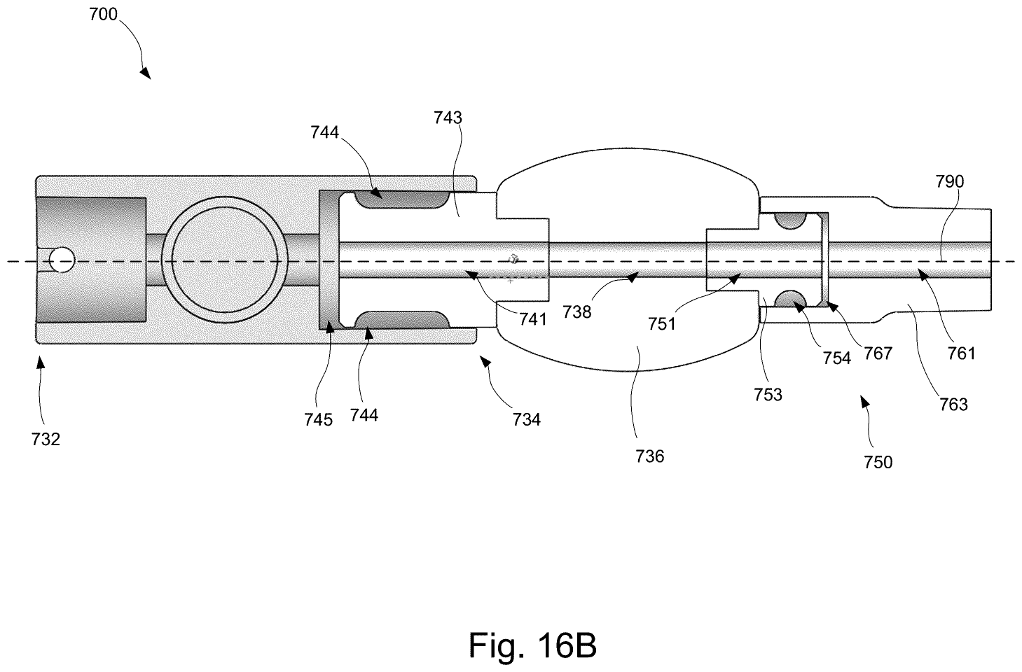

For example, referring to FIGS. 16A-16B, securement device 700 is illustrated with a coupling mechanism 750 for rotatably coupling the proximal end of handle 736 with protective sleeve 136 (not shown) in accordance with the present technology.

In one aspect, the coupling mechanism 750 may include cylindrical components 753, 763, and one or more pins 752. The distal end of component 753 is attached to handle 736 such that, when handle 736 is rotated, component 753 is rotated. The proximal portion of component 753 may include a circumferential slot 754 formed in the outer surface of component 753 and extending around the circumference of component 753.

Component 763 includes an interior cavity 767 at the distal end of component 763 that is configured to receive the proximal portion of component 753. The component 753 is rotatable within the cavity 767 (but such rotation need not translate to component 763) when handle 736 is rotated. Pins 752 extend from an interior surface forming the cavity 767 toward the axis 790 and into the slot 754. The pins 752 and slot 754 are configured to permit components 753, 763 to rotate about axis 790 relative to each other, but to prevent components 753, 763 from being separated longitudinally along axis 790 from each other.

The proximal portion of component 763 may be configured to couple with protective sleeve 136 (not shown). In some aspects, the proximal portion of component 763 has a smaller outer diameter than the distal portion of component 763. The distal end of sleeve 136 may be disposed over the exterior of the proximal portion of component 763 and coupled to component 763. The proximal portion of component 763 and protective sleeve 136 may be coupled or attached using any suitable technique. For example, protective sleeve 136 may be bonded to the proximal portion of component 763 or affixed thereto using adhesives, etc. Protective sleeve 136 may be removably coupled to the proximal portion of component 763 using any suitable mechanical coupling, such as a ring mounted on the proximal portion of component 763.

Component 753 includes an interior lumen 751 extending along the longitudinal axis 790 and component 763 includes an interior lumen 761 extending along the longitudinal axis 790. Lumens 751, 761 are configured to receive a portion of catheter 102 within each lumen. In one aspect, lumens 751, 761 each have the same diameter as lumen 738 (when handle 736 is not compressed) and lumen 741. As shown, lumens 741, 738, 751, 761 are disposed adjacent to each other along axis 790 and together form a larger lumen that receives catheter 102.

The securing device 700 shown in FIGS. 16A and 16B with coupling mechanism 750 may allow the protective sleeve 136 to be coupled to the distal end of the securement device 700 and thus prevent contamination of catheter 102 as it is advanced in the distal direction for insertion into the patient's vasculature. At the same time, coupling mechanism 750 permits handle 736 to be rotated relative to sleeve 136 such that sleeve 136 is not torqued and damaged when handle 736 is used to torque catheter 102.

In other aspects of the present technology, instead of modifying the securement device to aid in the user's ability to grip catheter 102, a gripping device that is separate from the securement device of the assembly may be provided with enhanced gripping features.

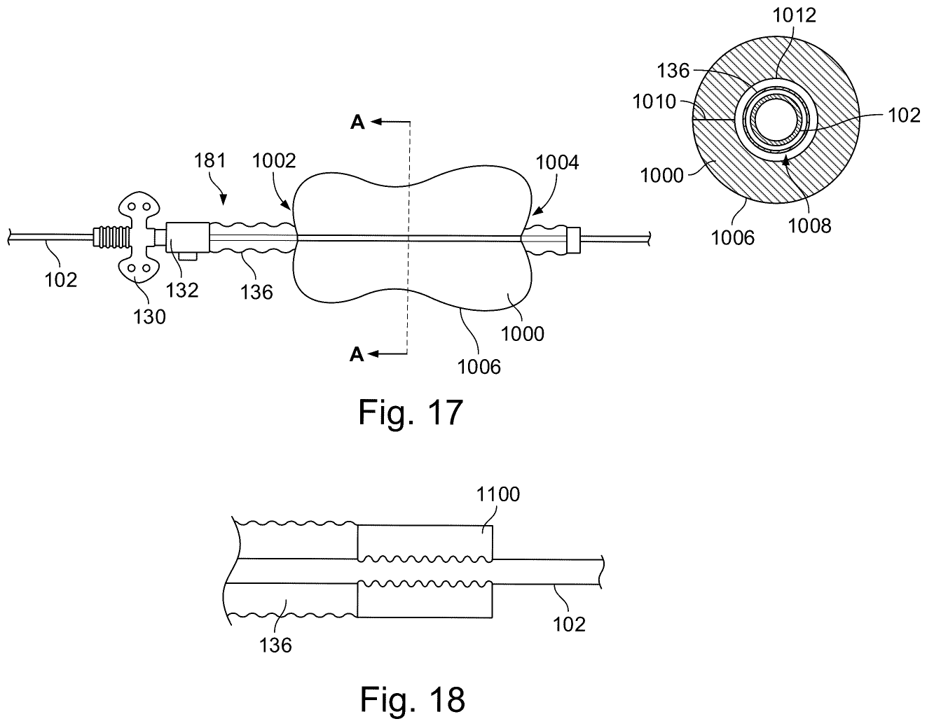

For example, referring to FIG. 17, in one aspect, a gripping device 1000 may be provided that is configured to grasp a portion of catheter 102, e.g., over protective sleeve 136 at portion 181 of catheter 102, exterior to the patient. In the aspect shown in FIG. 17, the gripping device 1000 may be configured as a handle that can be gripped by the user. For example, the gripping device 1000 includes a distal end 1002, a proximal end 1004, an exterior surface 1006, and an interior surface 1012 defining an interior lumen 1008 extending through the interior of gripping device 1000 from the distal end 1002 to the proximal end 1004. The interior lumen 1008 has a diameter selected to permit the interior lumen to receive a portion of catheter 102 and protective sleeve 136. In some aspects, the diameter of lumen 1008 is larger (e.g., in a range of 1% to 20% larger) than the outer diameter of catheter 102. In response to a gripping pressure (i.e., a compression force) applied to the exterior 1006 of gripping device 1000, gripping device 1000 is configured to deform such that the interior surface 1012 grips the exterior of sleeve 136 and catheter 102. The deformation of gripping device 1000 may decrease the diameter of at least a portion of the interior surface 1012 along at least one axis that is perpendicular to the longitudinal axis along which the interior lumen 1008 extends from the proximal end 1004 to the distal end 1002. In this way, at least a portion of the interior surface 1012 grips the exterior surface of catheter 102 such that any torque, pushing, or pulling force applied by a user to gripping device 1000 is transferred to catheter 102. Thus, by gripping and squeezing the gripping device 1000, a user may push, pull, or torque catheter 102 to position the blood pump 101 as desired within the patient. In some aspects, after the user stops gripping and squeezing gripping device 1000, gripping device 1000 is configured to return to its original, unstressed state/shape prior to deformation.

The gripping device 1000 may be made of rubber, silicone, or another material suitable for gripping objects and for being deformable, and in some aspects, reversibly deformable. The gripping device 1000 has a sufficient length from end 1004 to end 1002 to permit a stable grasping of gripping device 1000 and the catheter 102 (and/or sleeve 136) so that the user can push, pull, or torque catheter 102 to steer and position the pump 101 as desired in the patient. In some embodiments, the gripping device may be between 1 and 6 inches, although other suitable lengths may be chosen. In one aspect, handle 1000 may be configured to be removeable from sleeve 136 and catheter 102. In this regard, the gripping device may include an accessory attachable to the catheter. For example, handle 1000 may include a longitudinal slit 1010 (or a longitudinal gap) extending from a proximal to a distal end of handle 1000 that is configured such that the user can part the slit 1010 (or gap) to open the gripping device 1000 such that a portion of catheter 102 and/or sleeve 136 can be received by or removed from lumen 1008. As described above, with the catheter 102 and/or sleeve 136 received into the gripping device 1000, the user may grip the gripping device 1000 to apply sufficient gripping pressure (a compressive force) to the catheter 102 to torque, pull, or push the catheter as desired. In some aspects, the exterior 1006 of the gripping device 1000 may have an ergonomic shape (e.g., comprising concavely and/or convexly shaped portions of the exterior surface) to aid gripping.

Referring to FIG. 18, in another aspect, a gripping device 1100 is provided in accordance with the present technology. Gripping device 1100 may include any of the features described above in relation to gripping device 1000. As shown in FIG. 18, the gripping device 1100 may be disposed over catheter 102 (e.g., at portion 181 exterior to the patient) and configured to provide a stable gripping of the catheter 102 when a user applies gripping pressure (a compressive force) to the exterior of gripping device 1100. As described in relation to gripping device 1000, the gripping device 1100 may also be configured to deform in response to a compressive force applied by the user to the exterior of gripping device 1100 such that at least a portion of the internal lumen of gripping device 1100 deforms and the interior surface forming the lumen grips the exterior of catheter 102. In this way, when gripping the gripping device 1100, the user may push, pull, or torque to the catheter 102 to position the blood pump 101 appropriately within the patient. In one aspect, the gripping device 1100 may be integrated with sleeve 136. For example, in some aspects, the gripping member 1100 may be coupled to, attached, or integrated at an end (proximal or distal) of sleeve 136, as shown. In other aspects, gripping member 1100 may be integrated with sleeve 136 at a position between the ends of sleeve 136. The gripping member 1100 may be made of rubber, silicone, or any other suitable material. In one aspect, as shown in FIG. 18, the interior surface of gripping device 1100 that forms the interior lumen for receiving catheter 102 may be textured or include a pattern of raised portions configured to aid in gripping the exterior of catheter 102 when gripping device 1100 is gripped and deformed by the user.

It is to be appreciated that the interior lumens of gripping devices 1000, 1100 may each have a diameter when in an uncompressed state that is larger than the outer diameter of catheter 102 (and/or than the outer diameter of catheter 102 with sleeve 136 disposed over catheter 102). In this way, when devices 1000, 1100 are uncompressed (not deformed), devices 1000, 1100 and catheter 102 are slidable relative to each other with a portion of catheter 102 through the interior lumen of devices 1000, 1100.

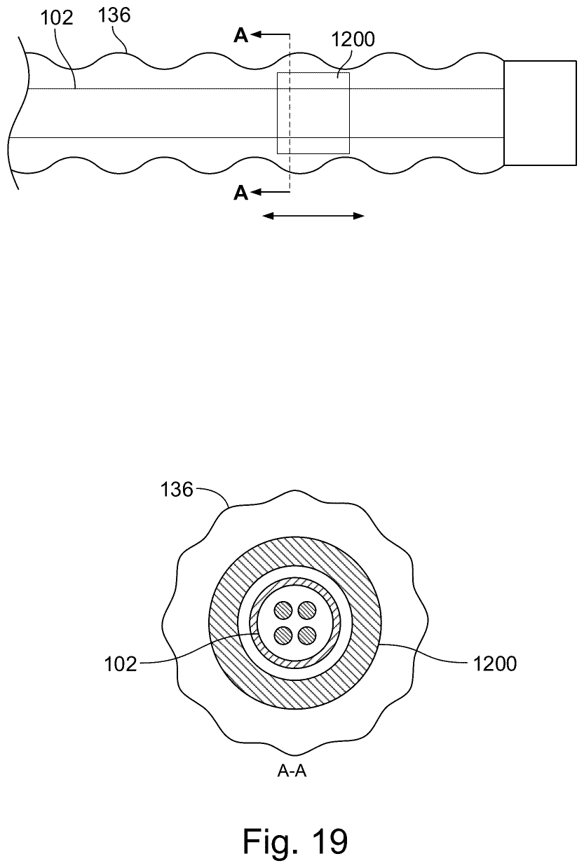

Referring to FIG. 19, in another aspect, a gripping device 1200 is provided in accordance with the present technology. Gripping device 1200 may include any of the features described above in relation to gripping devices 1000, 1100. In the aspect illustrated in FIG. 19, the gripping device 1200 is formed as a ring or tube with an interior lumen extending from the proximal end to the distal end of device 1200. Gripping device 1200 is disposed over the exterior of catheter 102 (e.g., at portion 181 of FIG. 17) and interior of sleeve 136. In one aspect, device 1200 is disposed between catheter 102 and sleeve 136 and the distal and proximal ends of sleeve 136 are coupled or fixed to the assembly 100, as described above. In one aspect, gripping device 1200 is configured to be freely slidable over catheter 102 when the user is not applying gripping or compression force to device 1200. In this regard, the inner diameter gripping member 1200 when uncompressed is larger than the outer diameter of catheter 102. Gripping member 1200 is configured such that, when gripping pressure is applied to gripping member 1200 by a user, the gripping member 1200 grips the exterior of catheter 102 and permits the user to push, pull, or torque the catheter 102 to position the blood pump 101 appropriately within the patient. The gripping member 1200 may be made of rubber, silicone, or any other suitable material.

From the foregoing and with reference to the various figure drawings, those skilled in the art will appreciate that certain modifications may also be made to the present disclosure without departing from the scope of the same. While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. A device for gripping a portion of a catheter of a catheter assembly, the device comprising:
a longitudinal axis;
a housing comprising a proximal end and a distal end, the housing extending along the longitudinal axis from the proximal end to the distal end of the housing, wherein the housing is configured to receive the catheter through an interior of the housing;
a securing mechanism configured to be selectively engaged to restrict or permit movement of the catheter through the housing; and
a grippable handle comprising a proximal end, a distal end, an exterior surface, and an interior surface defining an interior lumen,
wherein the grippable handle extends along the longitudinal axis from the proximal end to the distal end of the grippable handle,
wherein the interior lumen extends along the longitudinal axis and is configured to receive the portion of the catheter,
wherein the distal end of the grippable handle is rotatably coupled to the proximal end of the housing such that the grippable handle is rotatable relative to the housing about the longitudinal axis, and
wherein the grippable handle is configured to deform in response to a compression force applied to the grippable handle such that the interior surface grips the portion of the catheter to permit the grippable handle to transfer a torque force to the portion of the catheter.

2. The device of claim 1, wherein the grippable handle is configured to deform in response to a compression force applied to the exterior surface of the grippable handle.

3. The device of claim 1, further comprising an actuator configured to apply the compression force to the grippable handle.

4. The device of claim 1, wherein the distal end of the housing is configured to couple with a fixation device that is attachable to a patient.

5. The device of claim 4, wherein the distal end of the housing is configured to couple with the fixation device via a hemostasis valve disposed between the housing and the fixation device.

6. The device of claim 5, wherein the distal end of the housing includes a cavity configured to receive a proximal portion of the hemostasis valve.

7. The device of claim 1, wherein the distal end of the grippable handle is coupled to the proximal end of the housing such that the grippable handle is translatable relative to the housing along the longitudinal axis.

8. The device of claim 7, wherein, when the grippable handle is translated along the longitudinal axis and the interior surface grips the portion of the catheter, the grippable handle is configured to transfer a push or a pull force to the catheter.

9. The device of claim 1, further comprising a first coupling mechanism configured to rotatably couple the distal end of grippable handle to the proximal end of the housing.

10. The device of claim 9, wherein the first coupling mechanism is configured to couple the distal end of the grippable handle to the proximal end of the housing such that the grippable handle is translatable relative to the housing along the longitudinal axis.

11. The device of claim 9, further comprising a second coupling mechanism configured to rotatably couple the proximal end of the grippable handle to a protective sleeve of the catheter assembly.

12. The device of claim 1, wherein the grippable handle is made of a rubber or silicone material.

13. The device of claim 1, wherein a diameter of the interior lumen is larger than an outer diameter of the catheter when the grippable handle is not compressed.

14. The device of claim 13, wherein the device is slidable over the exterior of the catheter when the grippable handle is not compressed and the securing mechanism is engaged to permit movement of the catheter through the housing.

15. A catheter assembly, comprising:
a catheter; and
a gripping device comprising a proximal end, a distal end, an exterior surface, and an interior surface defining an interior lumen, the interior lumen extending from the proximal end to the distal end,
wherein the interior lumen is configured to receive a portion of the catheter, and
wherein the gripping device is configured to deform in response to a compression force applied to the exterior surface of the gripping device such that the interior surface grips the portion of the catheter to permit the gripping device to transfer a torque, push, and/or pull force to the portion of the catheter.

16. The catheter assembly of claim 15, wherein the gripping device is configured to deform in response to a compression force applied to the exterior surface of the gripping device.

17. The catheter assembly of claim 15, further comprising a sleeve disposed over the catheter.

18. The catheter assembly of claim 17, wherein the sleeve and the portion of the catheter extend through the interior lumen such that the gripping device is disposed over the sleeve.

19. The catheter assembly of claim 17, wherein the gripping device is coupled to the sleeve.

20. The catheter assembly of claim 19, wherein the gripping device is joined to the sleeve.

21. The catheter assembly of claim 19, wherein gripping device is coupled to one of a proximal end or a distal end of the sleeve.

22. The catheter assembly of claim 19, wherein the gripping device is coupled to a portion of the sleeve between a proximal end and a distal end of the sleeve.

23. The catheter assembly of claim 17, wherein the gripping device is disposed interior to the sleeve and exterior to the catheter.

24. The catheter assembly of claim 23, wherein the interior lumen is dimensioned such that that gripping device is slidable over the exterior of the catheter.

25. The catheter assembly of claim 15, wherein the interior lumen is dimensioned such that the gripping device is slidable over the exterior of the catheter when the gripping device is not compressed.

26. The catheter assembly of claim 15, wherein the exterior surface of the gripping device has an ergonomic shape.

27. The catheter assembly of claim 15, wherein the gripping device is made of a rubber or silicone material.

28. The catheter assembly of claim 15, wherein the catheter assembly is an intracardiac blood pump assembly.

29. The catheter assembly of claim 28, further comprising a blood pump comprising a proximal end and a distal end, wherein a distal end of the catheter is coupled to a proximal end of the blood pump.
